(19) United States
(12) Patent Application Publication
(10) Pub. No.: US 2025/0250567 A1
(43) Pub. Date: Aug. 7, 2025

(54) PROTOZOA TRANSCRIPTION FACTOR INHIBITOR

(71) Applicants: THE UNIVERSITY OF TOKYO, Tokyo (JP); OSAKA UNIVERSITY, Suita-shi, Osaka (JP)

(72) Inventors: Ken ISHII, Tokyo (JP); Hideo NEGISHI, Tokyo (JP); Cevayir COBAN, Tokyo (JP); Michelle Sue Jann LEE, Kikuchi (JP); Shiroh IWANAGA, Suita-shi (JP)

(73) Assignees: THE UNIVERSITY OF TOKYO, Tokyo (JP); OSAKA UNIVERSITY, Suita-shi, Osaka (JP)

(21) Appl. No.: 18/856,965

(22) PCT Filed: Apr. 14, 2023
(86) PCT No.: PCT/JP2023/015196

§ 371 Date: Oct. 15, 2024

(30) Foreign Application Priority Data
Apr. 15, 2022 (JP) 2022-067631

(51) Int. Cl. C12N 15/113

(52) U.S. Cl. CPC C12N 15/113; C12N 2310/11; C12N 2310/50

(57) ABSTRACT

An antiprotozoal agent targeting a transcription factor is disclosed. A pyrrole-imidazole polyamide (PIPA) specifically binding to a binding region of a protozoa transcription factor is disclosed. A method for producing the PIPA, and a protozoan transcription factor inhibitor that includes the PIPA are disclosed. Also disclosed is a method for treating or preventing a disease caused by a protozoon by inhibiting a protozoan morphological change using a PIPA that can function as a competitive pseudo-transcription factor specifically binding to a binding region for a protozoan transcription factor.

### TECHNICAL FIELD

The present disclosure relates to a pyrrole imidazole polyamide (PIPA) which specifically binds to a binding region for a protozoan transcription factor, a method for producing the PIPA, and a protozoan transcription factor inhibitor that includes the PIPA. More specifically, the present disclosure relates to a technique for treating or preventing a disease caused by a protozoon by inhibiting a protozoan morphological change using a PIPA that can function as a competitive pseudo-transcription factor specifically binding to a binding region for a protozoan transcription factor.

### BACKGROUND ART

Protozoan infections such as malaria have not yet been eradicated even in modern times, for example, malaria infections are still reported to kill 500,000 people a year worldwide. In addition, despite continued development of new drugs, drug-resistant strains emerge after a certain period of time since the development, making it difficult to achieve a radical cure thereof.

### SUMMARY OF INVENTION

### Solution to Problem

Protozoa are eukaryotes, but have a very distinctive transcriptional regulatory mechanism compared to mammalian cells. To date, no drugs have been developed that target a protozoan transcription factor. The present disclosure provides an antiprotozoal drug that targets a transcription factor by using a pyrrole imidazole polyamide (PIPA) that can function as a pseudo-transcription factor.

Therefore, the present disclosure provides the following items.
(Item 1)

A pyrrole imidazole polyamide (PIPA) which specifically binds to a binding region for a protozoan transcription factor.
(Item 2)

The PIPA which specifically binds to a binding region for a protozoan transcription factor according to the above item, wherein the protozoan transcription factor is a transcription factor specific to a protozoon.
(Item 3)

The PIPA according to any one of the above items, wherein the PIPA functions as a pseudo-transcription factor.
(Item 4)

The PIPA according to any one of the above items, wherein the PIPA has a binding affinity of about 500 nM or less as a dissociation constant (Kd value) for the binding region.
(Item 5)

The PIPA according to any one of the above items, wherein the PIPA has a hairpin structure or a cyclic structure, or two linear PIPAs are used in combination.
(Item 6)

The PIPA according to any one of the above items, wherein the PIPA inhibits a morphological change of the protozoon to at least a gametocyte.
(Item 7)

The PIPA according to any one of the above items, wherein the transcription factor includes an AP2 family transcription factor.

### (Item 8)

The PIPA according to any one of the above items, wherein the binding region includes 5′-TGCATG-3′ (SEQ ID NO: 1) or an altered sequence thereof, the altered sequence including a sequence wherein any one base in 5′-TGCATG-3′ (SEQ ID NO: 1) is deleted or mutated and a sequence wherein one base is added at any position in 5′-TGCATG-3′ (SEQ ID NO: 1).

### (Item 9)

The PIPA according to any one of the above items, wherein the binding region includes NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.

### (Item 10)

The PIPA according to any one of the above items, wherein the binding region includes 5′-TGCACT-3′ (SEQ ID NO: 8) or an altered sequence thereof, the altered sequence including a sequence wherein any one base in 5′-TGCACT-3′ (SEQ ID NO: 8) is deleted or mutated and a sequence wherein one base is added at any position in 5′-TGCACT-3′ (SEQ ID NO: 8).

### (Item 11)

The PIPA according to any one of the above items, wherein the binding region includes NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.

### (Item 12)

The PIPA according to any one of the above items, wherein the PIPA includes the following structure:

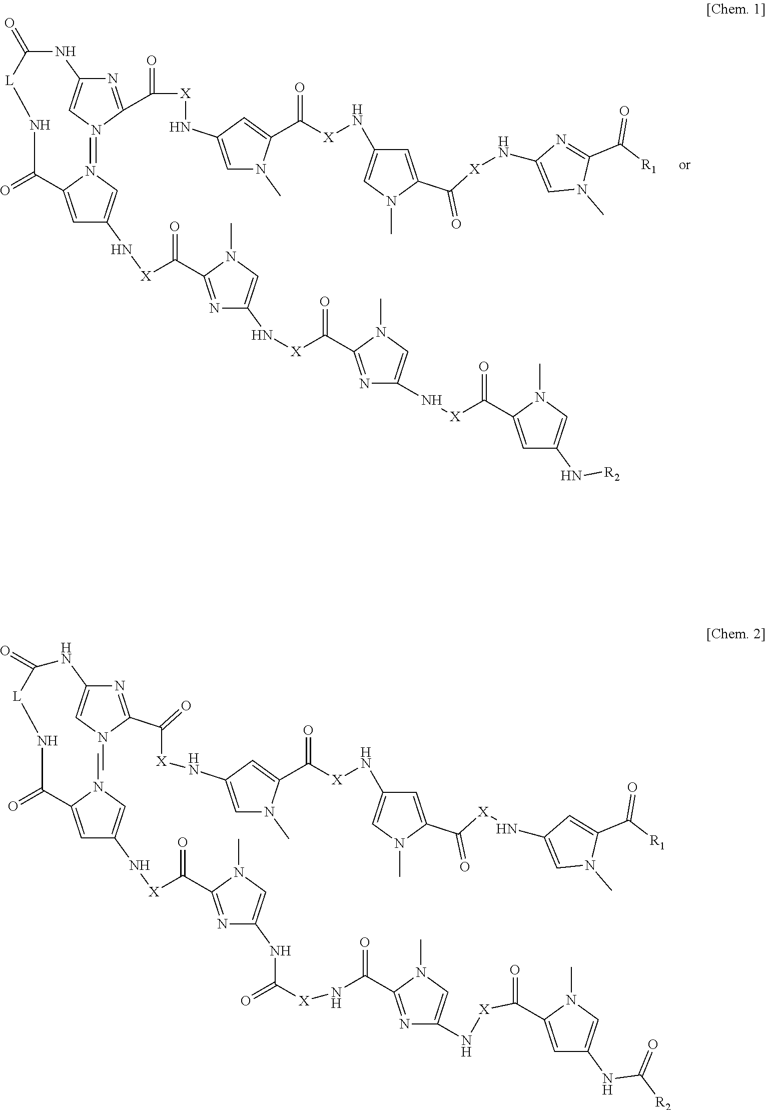

where
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.
(Item 13)

The PIPA according to any one of the above items, wherein the aliphatic amino acid residue includes a molecule having an amino group and a carboxy group.
(Item 14)

The PIPA according to any one of the above items, wherein the aliphatic amino acid residue includes glycine, β-alanine, γ-aminobutyric acid, R2,4-diaminobutyric acid, and 5-aminovaleric acid.
(Item 15)

The PIPA according to any one of the above items, wherein the PIPA has the following structure:

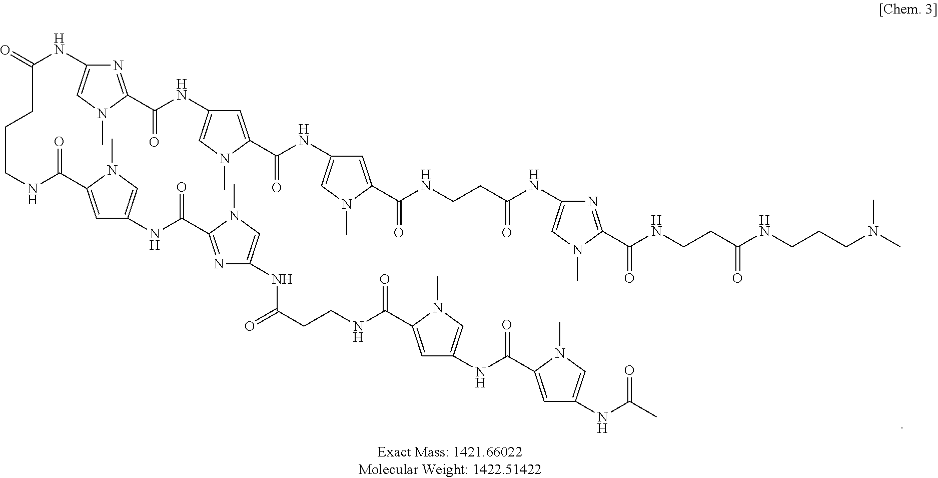 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

(Item 16)

The PIPA according to any one of the above items, wherein the PIPA has the following structure:

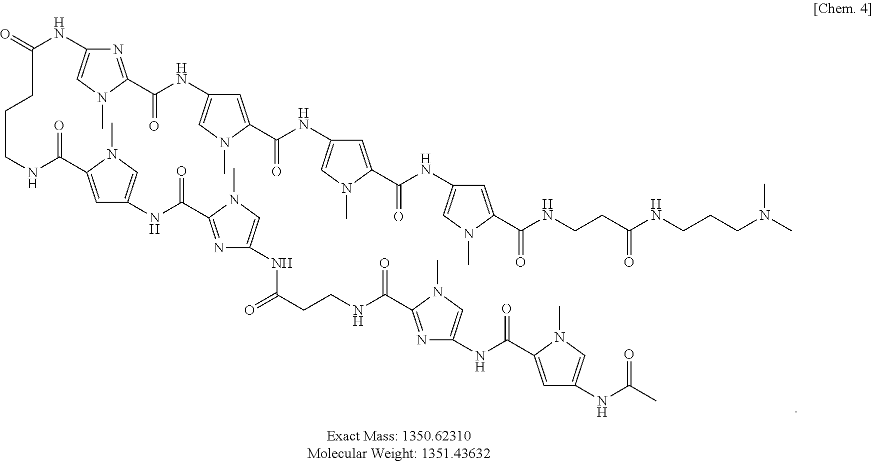 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

(Item 17)

The PIPA according to any one of the above items, wherein the protozoon includes Plasmodium spp., Leishmania spp., Toxoplasma spp., Cryptosporidium spp., and a coccidium.
(Item A1)

A pyrrole imidazole polyamide (PIPA) which specifically binds to a binding region for a protozoan transcription factor, for inhibiting a function of the protozoan transcription factor.
(Item A2)

The PIPA according to any one of the above items, wherein the protozoan transcription factor is a transcription factor specific to a protozoon.
(Item A3)

The PIPA according to any one of the above items, wherein the PIPA functions as a pseudo-transcription factor.
(Item A4)

The PIPA according to any one of the above items, wherein the PIPA has a binding affinity of about 500 nM or less as a dissociation constant (Kd value) for the binding region.
(Item A5)

The PIPA according to any one of the above items, wherein the PIPA has a hairpin structure or a cyclic structure, or two linear PIPAs are used in combination.
(Item A6)

The PIPA according to any one of the above items, wherein the PIPA inhibits a morphological change of the protozoon to at least a gametocyte.
(Item A7)

The PIPA according to any one of the above items, wherein the transcription factor includes an AP2 family transcription factor.
(Item A8)

The PIPA according to any one of the above items, wherein the binding region includes 5′-TGCATG-3′ (SEQ ID NO: 1) or an altered sequence thereof, the altered sequence including a sequence wherein any one base in 5′-TGCATG-3′ (SEQ ID NO: 1) is deleted or mutated and a sequence wherein one base is added at any position in 5′-TGCATG-3′ (SEQ ID NO: 1).
(Item A9)

The PIPA according to any one of the above items, wherein the binding region includes NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.
(Item A10)

The PIPA according to any one of the above items, wherein the binding region includes 5′-TGCACT-3′ (SEQ ID NO: 8) or an altered sequence thereof, the altered sequence including a sequence wherein any one base in 5′-TGCACT-3′ (SEQ ID NO: 8) is deleted or mutated and a sequence wherein one base is added at any position in 5′-TGCACT-3′ (SEQ ID NO: 8).
(Item A11)

The PIPA according to any one of the above items, wherein the binding region includes NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.
(Item A12)

The PIPA according to any one of the above items, wherein the PIPA has the following structure:

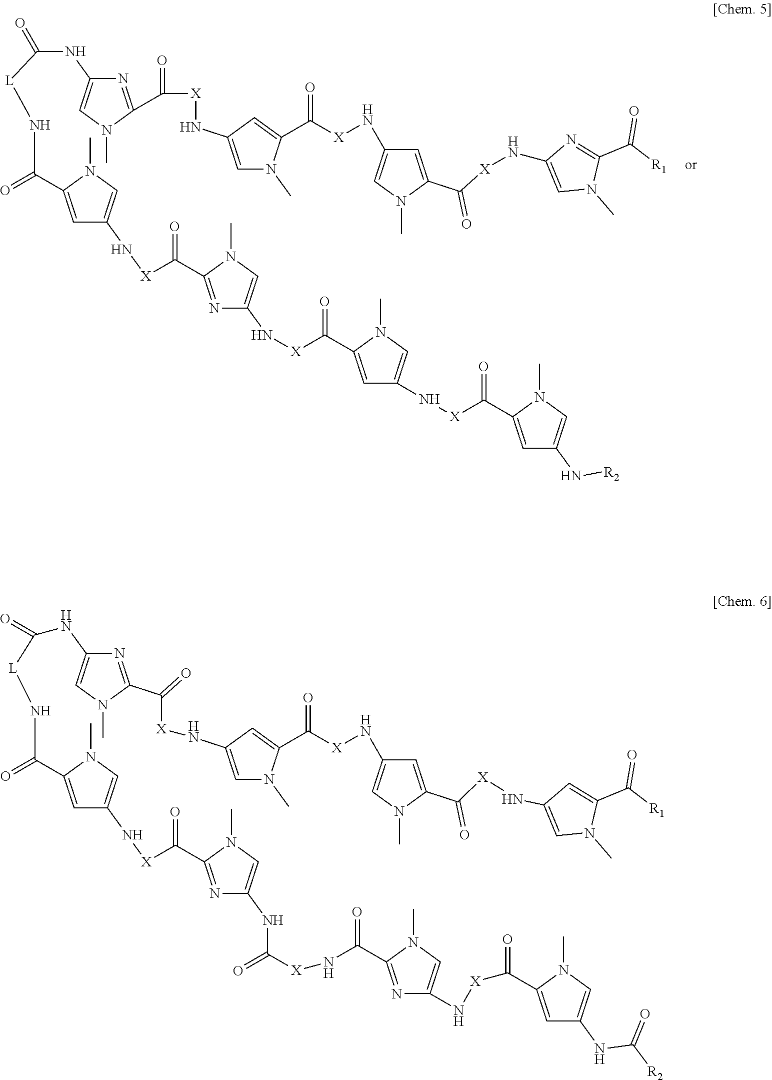

where
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.
(Item A13)

The PIPA according to any one of the above items, wherein the aliphatic amino acid residue includes a molecule having an amino group and a carboxy group.
(Item A14)

The PIPA according to any one of the above items, wherein the aliphatic amino acid residue includes glycine, β-alanine, γ-aminobutyric acid, R2,4-diaminobutyric acid, and 5-aminovaleric acid.
(Item A15)

The PIPA according to any one of the above items, wherein the PIPA has the following structure:

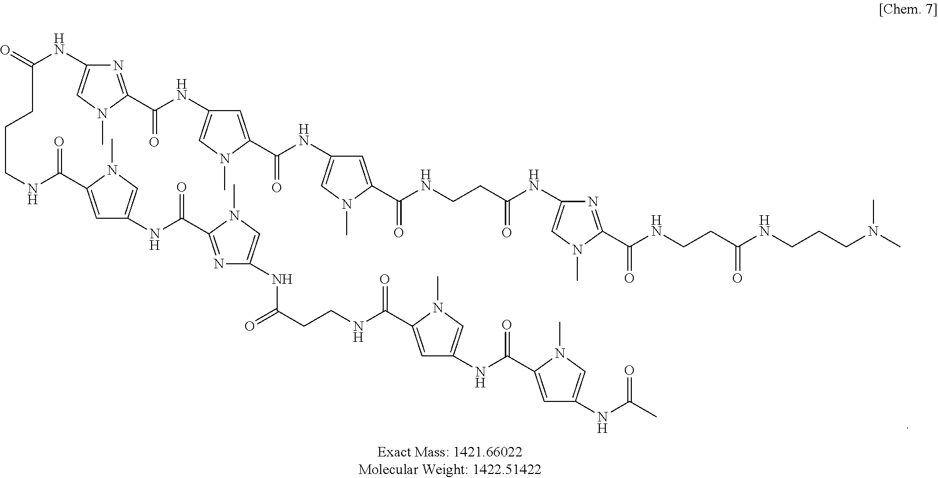 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

(Item A16)

The PIPA according to any one of the above items, wherein the PIPA has the following structure:

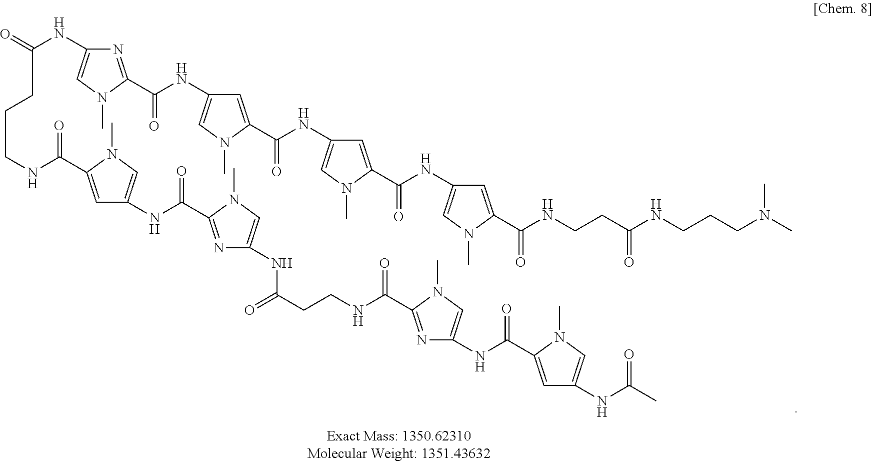 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

(Item A17)

The PIPA according to any one of the above items, wherein the protozoon includes Plasmodium spp., Leishmania spp., Toxoplasma spp., Cryptosporidium spp., and a coccidium.
(Item A1A)

A pyrrole imidazole polyamide (PIPA) which specifically binds to a binding region for a protozoan transcription factor, for use as a pseudo-transcription factor.
(Item A1B)

A composition including
a pyrrole imidazole polyamide (PIPA) which specifically binds to a binding region for a protozoan transcription factor, for inhibiting a function of the protozoan transcription factor.
(Item A1C)

A composition including
a pyrrole imidazole polyamide (PIPA) which specifically binds to a binding region for a protozoan transcription factor, for use as a pseudo-transcription factor.
(Item A1D)

A method for inhibiting a function of a protozoan transcription factor in a subject, the method including
contacting the protozoan transcription factor in the subject with an effective amount of a pyrrole imidazole polyamide (PIPA) which specifically binds to a binding region for the transcription factor.
(Item A1E)

A method for using a pyrrole imidazole polyamide (PIPA) as a pseudo-transcription factor in a subject, the method including
applying to the subject an effective amount of a PIPA which specifically binds to a binding region for a protozoan transcription factor.
(Item B1)

A protozoan transcription factor inhibitor including
a pyrrole imidazole polyamide (PIPA) which specifically binds to a binding region for a protozoan transcription factor.
(Item B2)

The protozoan transcription factor inhibitor according to any one of the above items, wherein the protozoan transcription factor is a transcription factor specific to a protozoon.
(Item B3)

The protozoan transcription factor inhibitor according to any one of the above items, wherein the protozoan transcription factor inhibitor functions as a pseudo-transcription factor.
(Item B4)

The protozoan transcription factor inhibitor according to any one of the above items, wherein the protozoan transcription factor inhibitor has a binding affinity of about 500 nM or less as a dissociation constant (Kd value) for the binding region.
(Item B5)

The protozoan transcription factor inhibitor according to any one of the above items, wherein the PIPA has a hairpin structure or a cyclic structure, or two linear PIPAs are used in combination.
(Item B6)

The protozoan transcription factor inhibitor according to any one of the above items, wherein the protozoan transcription factor inhibitor inhibits a morphological change of the protozoon to at least a gametocyte.
(Item B7)

The protozoan transcription factor inhibitor according to any one of the above items, wherein the transcription factor includes an AP2 family transcription factor.
(Item B8)

The protozoan transcription factor inhibitor according to any one of the above items, wherein the binding region includes 5′-TGCATG-3′ (SEQ ID NO: 1) or an altered sequence thereof, the altered sequence including a sequence wherein any one base in 5′-TGCATG-3′ (SEQ ID NO: 1) is deleted or mutated and a sequence wherein one base is added at any position in 5′-TGCATG-3′ (SEQ ID NO: 1).
(Item B9)

The protozoan transcription factor inhibitor according to any one of the above items, wherein the binding region includes NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.
(Item B10)

The protozoan transcription factor inhibitor according to any one of the above items, wherein the binding region includes 5′-TGCACT-3′ (SEQ ID NO: 8) or an altered sequence thereof, the altered sequence including a sequence wherein any one base in 5′-TGCACT-3′ (SEQ ID NO: 8) is deleted or mutated and a sequence wherein one base is added at any position in 5′-TGCACT-3′ (SEQ ID NO: 8).
(Item B11)

The protozoan transcription factor inhibitor according to any one of the above items, wherein the binding region includes NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.
(Item B12)

The protozoan transcription factor inhibitor according to any one of the above items, wherein the PIPA includes the following structure:

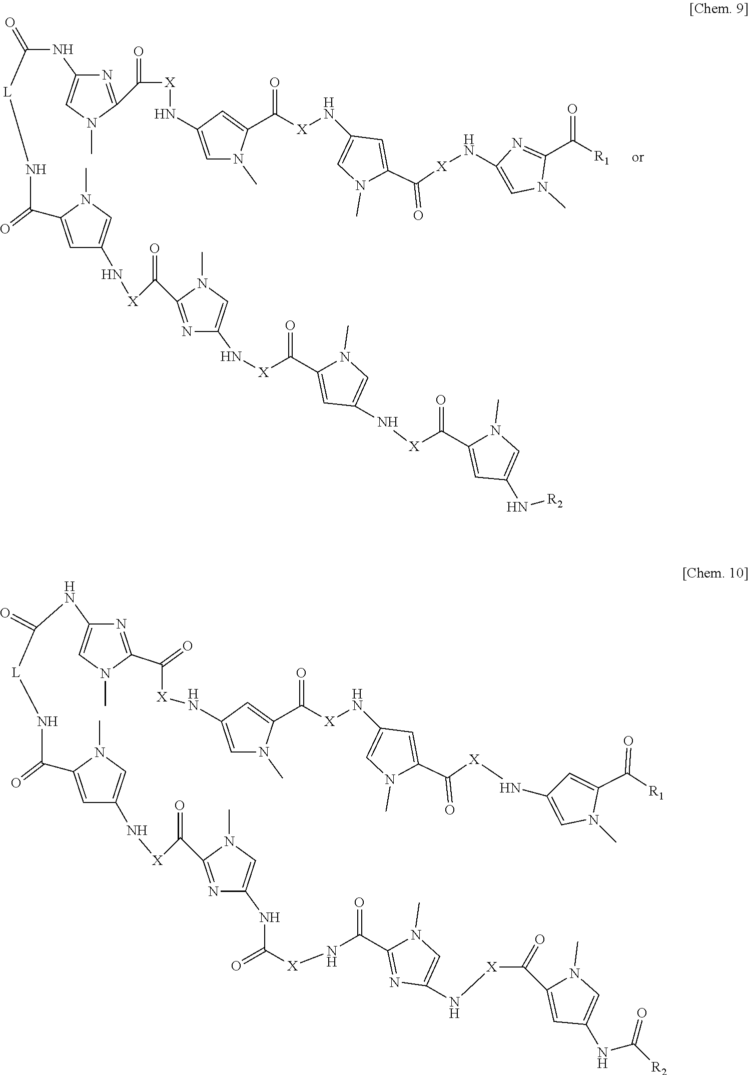

where
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.
(Item B13)

The protozoan transcription factor inhibitor according to any one of the above items, wherein the aliphatic amino acid residue includes a molecule having an amino group and a carboxy group.
(Item B14)

The protozoan transcription factor inhibitor according to any one of the above items, wherein the aliphatic amino acid residue includes glycine, β-alanine, γ-aminobutyric acid, R2,4-diaminobutyric acid, and 5-aminovaleric acid.
(Item B15)

The protozoan transcription factor inhibitor according to any one of the above items, wherein the PIPA has the following structure:

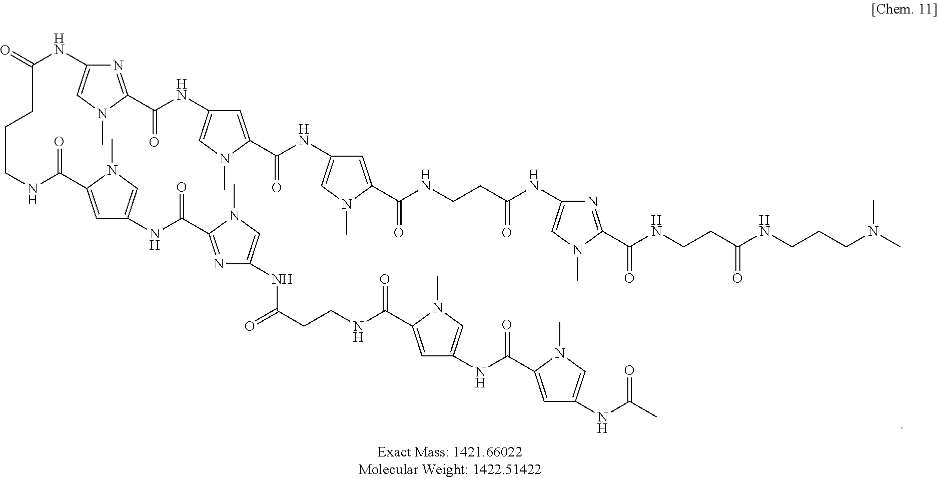 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

(Item B16)

The protozoan transcription factor inhibitor according to any one of the above items, wherein the PIPA has the following structure:

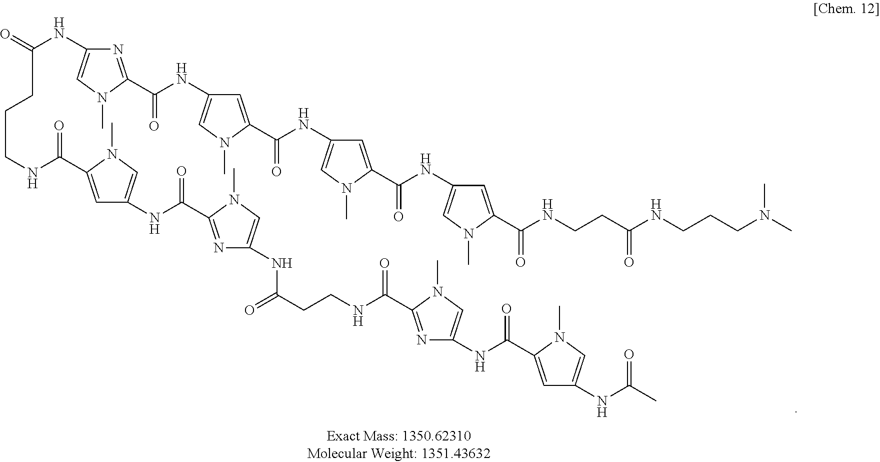 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

(Item B17)

The protozoan transcription factor inhibitor according to any one of the above items, wherein the protozoon includes Plasmodium spp., Leishmania spp., Toxoplasma spp., Cryptosporidium spp., and a coccidium.
(Item C1)

A method for producing a protozoan transcription factor inhibitor including a pyrrole imidazole polyamide (PIPA), the method including:
providing a binding region for a protozoan transcription factor; and
designing the PIPA to specifically bind to the binding region.
(Item C2)

The method according to the above item, wherein the designing includes linking a pyrrole and/or an imidazole selected to correspond to a nucleotide sequence of the binding region and, if necessary, replacing one or more pyrroles or imidazoles in the linked pyrrole and/or imidazole molecule with β-alanine.
(Item D1)

A therapeutic or prophylactic agent for a disease caused by a protozoon, the agent including
a protozoan transcription factor including a pyrrole imidazole polyamide (PIPA) which specifically binds to a binding region for the protozoan transcription factor.
(Item D2)

The therapeutic or prophylactic agent according to any one of the above items, wherein the protozoan transcription factor is a transcription factor specific to a protozoon.
(Item D3)

The therapeutic or prophylactic agent according to any one of the above items, wherein the therapeutic or prophylactic agent functions as a pseudo-transcription factor.
(Item D4)

The therapeutic or prophylactic agent according to any one of the above items, wherein the therapeutic or prophylactic agent has a binding affinity of about 500 nM or less as a dissociation constant (Kd value) for the binding region.
(Item D5)

The therapeutic or prophylactic agent according to any one of the above items, wherein the PIPA has a hairpin structure or a cyclic structure, or two linear PIPAs are used in combination.
(Item D6)

The therapeutic or prophylactic agent according to any one of the above items, wherein the therapeutic or prophylactic agent inhibits a morphological change of the protozoon to at least a gametocyte.
(Item D7)

The therapeutic or prophylactic agent according to any one of the above items, wherein the transcription factor includes an AP2 family transcription factor.
(Item D8)

The therapeutic or prophylactic agent according to any one of the above items, wherein the binding region includes 5′-TGCATG-3′ (SEQ ID NO: 1) or an altered sequence thereof, the altered sequence including a sequence wherein any one base in 5′-TGCATG-3′ (SEQ ID NO: 1) is deleted or mutated and a sequence wherein one base is added at any position in 5′-TGCATG-3′ (SEQ ID NO: 1).
(Item D9)

The therapeutic or prophylactic agent according to any one of the above items, wherein the binding region includes NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.
(Item D10)

The therapeutic or prophylactic agent according to any one of the above items, wherein the binding region includes 5′-TGCACT-3′ (SEQ ID NO: 8) or an altered sequence thereof, the altered sequence including a sequence wherein any one base in 5′-TGCACT-3′ (SEQ ID NO: 8) is deleted or mutated and a sequence wherein one base is added at any position in 5′-TGCACT-3′ (SEQ ID NO: 8).
(Item D11)

The therapeutic or prophylactic agent according to any one of the above items, wherein the binding region includes NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.
(Item D12)

The therapeutic or prophylactic agent according to any one of the above items, wherein the PIPA includes the following structure:

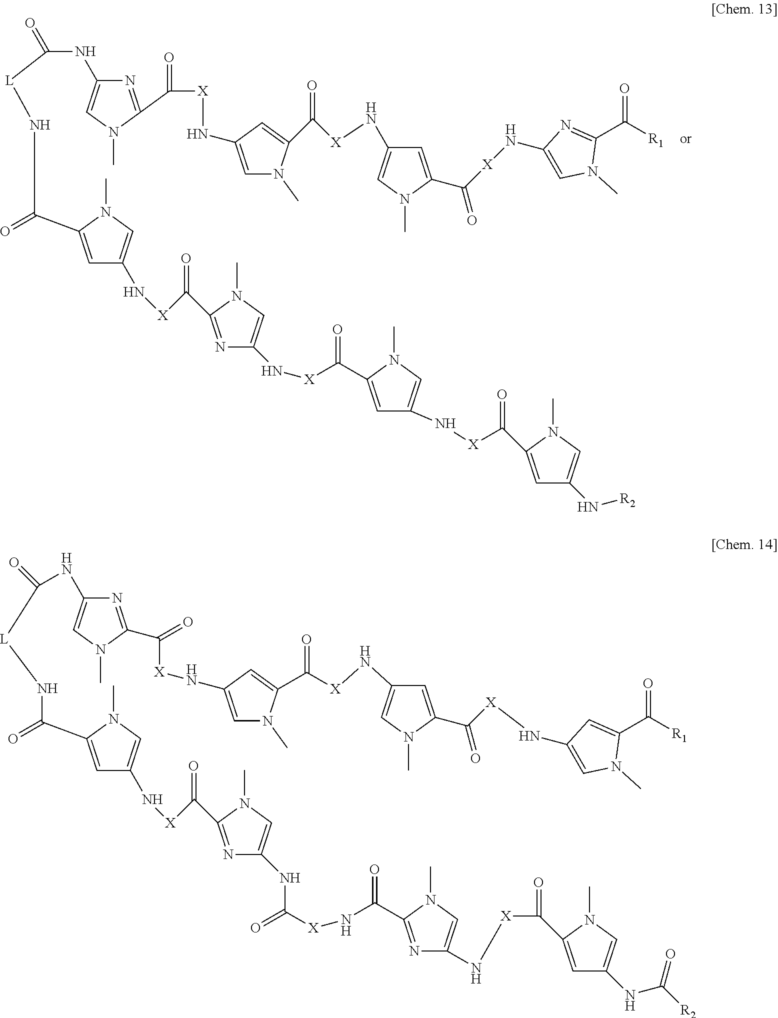

where
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.
(Item D13)

The therapeutic or prophylactic agent according to any one of the above items, wherein the aliphatic amino acid residue includes a molecule having an amino group and a carboxy group.
(Item D14)

The therapeutic or prophylactic agent according to any one of the above items, wherein the aliphatic amino acid residue includes glycine, β-alanine, γ-aminobutyric acid, R2,4-diaminobutyric acid, and 5-aminovaleric acid.
(Item D15)

The therapeutic or prophylactic agent according to any one of the above items, wherein the PIPA has the following structure:

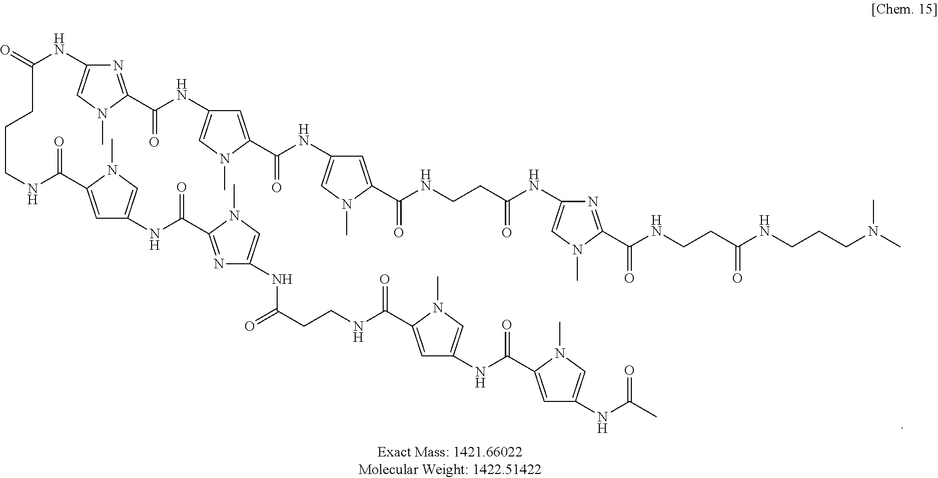 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

(Item D16)

The therapeutic or prophylactic agent according to any one of the above items, wherein the PIPA has the following structure:

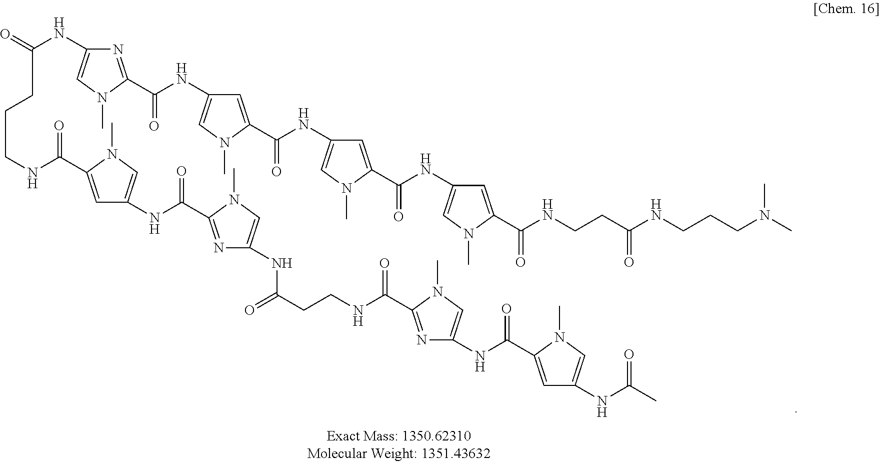 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

(Item D17)

The therapeutic or prophylactic agent according to any one of the above items, wherein the protozoon includes Plasmodium spp., Leishmania spp., Toxoplasma spp., Cryptosporidium spp., and a coccidium.
(Item D1A)

A pyrrole imidazole polyamide (PIPA) which specifically binds to a binding region for a protozoan transcription factor, for treating or preventing a disease caused by a protozoon.
(Item E1)

A method for treating or preventing a disease caused by protozoon in a subject, the method including
administering to the subject an effective amount of a protozoan transcription factor inhibitor, the protozoan transcription factor inhibitor including a pyrrole imidazole polyamide (PIPA) which specifically binds to a binding region for a protozoan transcription factor.
(Item X1)

A conjugate including:
pyrrole imidazole polyamide (PIPA) which specifically binds to a binding region for a protozoan transcription factor; and
a protozoon-specific factor that is different from the PIPA.
(Item X2)

The conjugate according to any one of the items above, wherein the protozoon-specific factor includes a factor that binds to a surface protein of a malaria-infected red blood cell.
(Item X3)

The conjugate according to any one of the above items, wherein the protozoon-specific factor has a proliferation inhibitory effect on Plasmodium spp., Leishmania spp., Toxoplasma spp., Cryptosporidium spp., and a coccidium.
(Item X4)

The conjugate according to any one of the above items, wherein the PIPA and the protozoon-specific factor are linked by a linker.
(Item X5)

The conjugate according to any one of the above items, wherein the linker is a C1-6 alkyl linker.
(Item X6)

The conjugate according to any one of the above items, wherein the PIPA and the protozoon-specific factor are directly linked.
(Item X7)

The conjugate according to any one of the above items, wherein the protozoon-specific factor includes a pyridazinone derivative.
(Item X8)

The conjugate according to any one of the above items, wherein the pyridazinone derivative is MBX-4055 represented by the following formula:

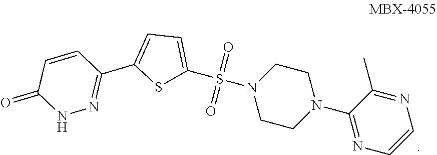 [SMILES: Cc1nccnc1N1CCN(S(=O)(=O)c2ccc(-c3ccc(=O)[nH]n3)s2)CC1]

(Item X9)

The conjugate according to any one of the above items, wherein the pyridazinone derivative is an MBX-4055 derivative represented by the following formula:

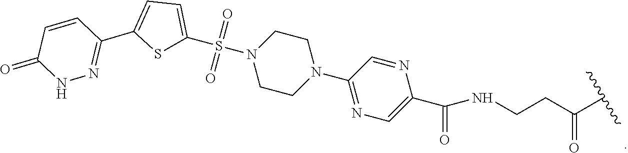 [SMILES: *C(=O)CCNC(=O)c1cnc(N2CCN(S(=O)(=O)c3ccc(-c4ccc(=O)[nH]n4)s3)CC2)cn1]

(Item X10)

The conjugate according to any one of the above items, wherein the pyridazinone derivative is an MBX-4055 derivative represented by the following formula:

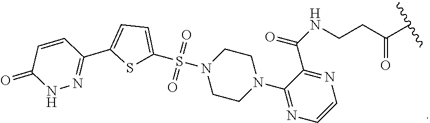 [SMILES: *C(=O)CCNC(=O)c1nccnc1N1CCN(S(=O)(=O)c2ccc(-c3ccc(=O)[nH]n3)s2)CC1]

(Item X11)

The conjugate according to any one of the above items, wherein the pyridazinone derivative is an MBX-4055 derivative represented by the following formula:

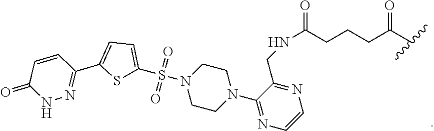 [SMILES: *C(=O)CCCC(=O)NCc1nccnc1N1CCN(S(=O)(=O)c2ccc(-c3ccc(=O)[nH]n3)s2)CC1]

(Item X12)

The conjugate according to any one of the above items, wherein the protozoan transcription factor is a transcription factor specific to a protozoon.
(Item X13)

The conjugate according to any one of the above items, wherein the conjugate functions as a pseudo-transcription factor.
(Item X14)

The conjugate according to any one of the above items, wherein the conjugate has a binding affinity of about 500 nM or less as a dissociation constant (Kd value) for the binding region.
(Item X15)

The protozoan transcription factor inhibitor according to any one of the above items, wherein the PIPA has a hairpin structure or a cyclic structure, or two linear PIPAs are used in combination.
(Item X16)

The conjugate according to any one of the above items, wherein the conjugate inhibits a morphological change of the protozoon to at least a gametocyte.
(Item X17)

The conjugate according to any one of the above items, wherein the transcription factor includes an AP2 family transcription factor.
(Item X18)

The conjugate according to any one of the above items, wherein the binding region includes 5′-TGCATG-3′ (SEQ ID NO: 1) or an altered sequence thereof, the altered sequence including a sequence wherein any one base in 5′-TGCATG-3′ (SEQ ID NO: 1) is deleted or mutated and a sequence wherein one base is added at any position in 5′-TGCATG-3′ (SEQ ID NO: 1).
(Item X19)

The conjugate according to any one of the above items, wherein the binding region includes NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.
(Item X20)

The conjugate according to any one of the above items, wherein the binding region includes 5′-TGCACT-3′ (SEQ ID NO: 8) or an altered sequence thereof, the altered sequence including a sequence wherein any one base in 5′-TGCACT-3′ (SEQ ID NO: 8) is deleted or mutated and a sequence wherein one base is added at any position in 5′-TGCACT-3′ (SEQ ID NO: 8).
(Item X21)

The conjugate according to any one of the above items, wherein the binding region includes NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.
(Item X22)

The conjugate according to any one of the above items, wherein the PIPA includes the following structure:

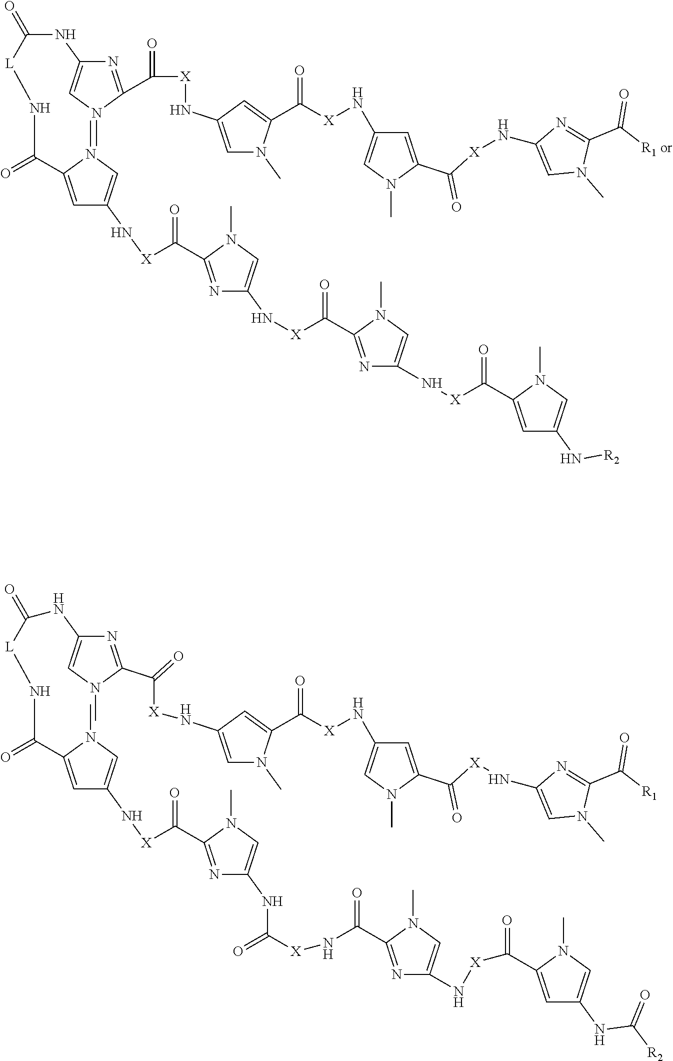

where
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.
(Item X23)

The conjugate according to any one of the above items, wherein the aliphatic amino acid residue includes a molecule having an amino group and a carboxy group.
(Item X24)

The conjugate according to any one of the above items, wherein the aliphatic amino acid residue includes glycine, β-alanine, γ-aminobutyric acid, R2,4-diaminobutyric acid, and 5-aminovaleric acid.
(Item X25)

The conjugate according to any one of the above items, wherein the PIPA has the following structure:

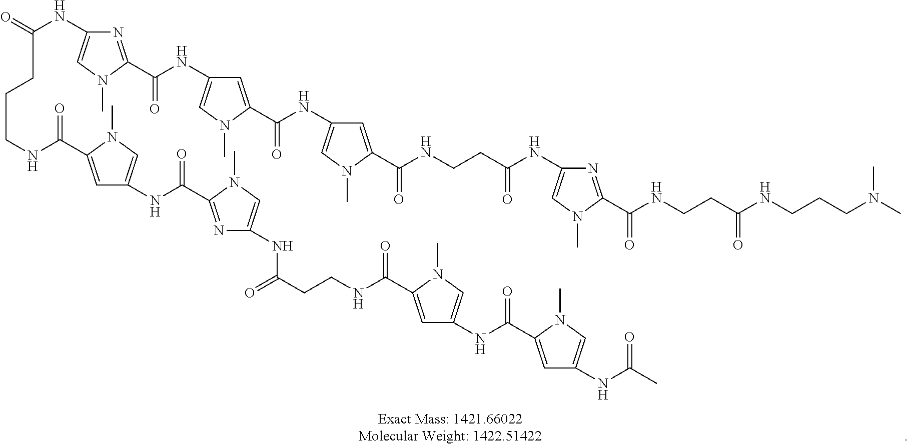 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

(Item X26)

The conjugate according to any one of the above items, wherein the PIPA has the following structure:

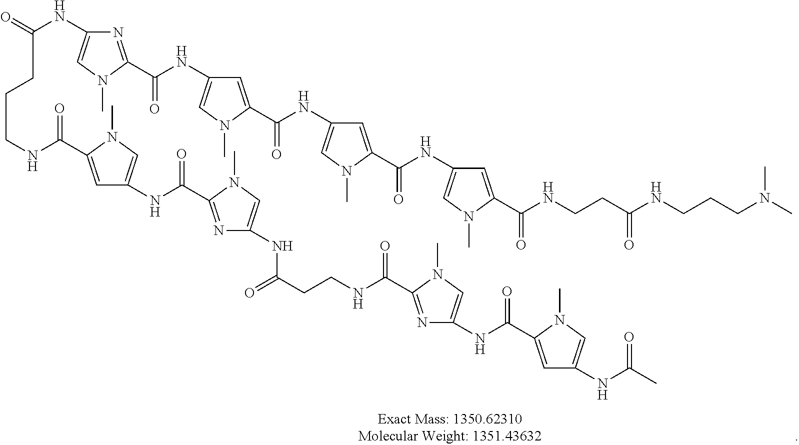 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

(Item X27)

The conjugate according to any one of the above items, wherein the protozoon includes Plasmodium spp., Leishmania spp., Toxoplasma spp., Cryptosporidium spp., and a coccidium.

In the present disclosure, it is intended that the above-mentioned one or more features may be provided in further combinations in addition to the explicit combinations. Note that, those skilled in the art will be recognized further embodiments and advantages of the present disclosure upon reading and understanding the following detailed description, if necessary.

Note that, features and significant actions and effects of the present disclosure other than those described above will become clear to those skilled in the art by referring to the following embodiments of the invention and drawings.

### Advantageous Effects of Invention

The present disclosure can provide a PIPA which specifically binds to a binding region for a protozoan transcription factor. This can provide a therapeutic or prophylactic agent for diseases caused by protozoa which are still difficult to achieve radical cure and an innovative and powerful drug for eradicating protozoan infections.

### BRIEF DESCRIPTION OF DRAWINGS

FIG. 1a FIG. 1a shows the sequences, the structural formulae, and the molecular weights of PIPAs according to one embodiment of the present disclosure. AP2-PIPA1 (left, FIG. 1a) and AP2-PIPA2 (right, FIG. 1a) used in Example 1 are shown;

FIG. 1b is a schematic diagram showing a DNA fragment used in one embodiment of the present disclosure to analyze a binding strength of AP2-PIPA1 to its target sequence;

FIG. 1c shows a graph showing analysis results of a binding strength (Kd value) of AP2-PIPA1 to its target sequence in one embodiment of the present disclosure;

FIG. 2a shows graphs showing analysis results of cytotoxicity (DNA release in a culture supernatant) of AP2-PIPA1 in one embodiment of the present disclosure;

FIG. 2b shows graphs showing analysis results of cytotoxicity (LDH activity in a culture supernatant) of AP2-PIPA1 in one embodiment of the present disclosure;

FIG. 3a shows micrographs showing analysis results of malaria inhibitory effects of AP2-PIPA1 and AP2-PIPA2 in cells in one embodiment of the present disclosure;

FIG. 3b shows a description of an analysis (FACS analysis) of malaria inhibitory effects of AP2-PIPA1 and AP2-PIPA2 in cells in one embodiment of the present disclosure;

FIG. 3c shows graphs showing analysis (FACS analysis) results of malaria inhibitory effects of AP2-PIPA1 and AP2-PIPA2 in cells in one embodiment of the present disclosure;

FIG. 4 shows graphs showing analysis results of inhibitory effects of AP2-PIPA1 and AP2-PIPA2 on malaria drug-resistant strains in cells in one embodiment of the present disclosure. Plasmodia were cultured in red blood cells in the presence of various concentrations of a PIPA and, 72 hours later, parasitemia was assessed;

FIG. 5a shows analysis results of toxicity of AP2-PIPA1 in mice in one embodiment of the present disclosure. Toxicity of a single intraperitoneal administration of AP2-PIPA1 was studied in mice. Doses were 0 (PBS), 5, 10, and 20 mg/kg, and the mice were observed for 7 days after administration. Observation of viability and general conditions, measurement of body weight, a hematology test, a blood chemistry test, and necropsy were performed as toxicity assessment indices. No deaths were observed, and no changes that were considered to be abnormal were found in the observation of general conditions, a weight change, the hematology test, the blood chemistry test, or the autopsy. Significant increases in AST activity were seen in the 5 and 20 mg/kg dose groups. For the 10 mg/kg dose group, although the difference was not significant, an upward trend was observed. However, the value for each individual in these groups falls within a background value (34 to 58 IU/L) and thus the changes are determined to be due to physiological variation. Based on these results, the minimum lethal dose of AP2-PIPA1 in this study was estimated to be greater than 20 mg/kg;

FIG. 5b shows analysis results of toxicity of AP2-PIPA1 in mice in one embodiment of the present disclosure. Mice received various doses of AP2-PIPA1 once daily for 7 days orally or intraperitoneally. On day 8, blood was collected and AST and ALT were measured. Significant difference tests were performed for the 3 mg/kg and 10 mg/kg dose groups relative to the control group for each route of administration. As a result of the Bartlett's test for Equality of variances, p>0.01 was determined to be equal variance, and as a result of Dunnett's two-tailed test, there was no significant difference at p>0.05;

FIG. 6 shows an analysis of a malaria inhibitory effect of AP2-PIPA1 in mice in one embodiment of the present disclosure;

FIG. 7a shows the sequence, the structural formula, and the molecular weight of the PIPA that targets TGCATG in one embodiment of the present disclosure (arrangement of β-alanine was altered). Arrangement of β-alanine was altered at two positions in AP2-PIPA1;

FIG. 7b shows the sequence, the structural formula, and the molecular weight of the PIPA that targets TGCATG in one embodiment of the present disclosure (pyrroles for one base pair were added). Since AP2-PIPA1 uses γAbu which has an affinity for an A-T pair in a hairpin structure, a Py-Py pair that recognizes the A-T pair was added. If a recognition sequence is increased and a GC/CG pair is at a 5′ end, the γAbu is not involved in binding;

FIG. 7c shows the sequence, the structural formula, and the molecular weight of the PIPA that targets TGCATG in one embodiment of the present disclosure (hairpin structure was converted from γAbu to D-Dab). Since AP2-PIPA1 uses γAbu which has an affinity for an A-T pair in a hairpin structure, this hairpin structure was converted to D-Dab. The D-Dab also has an affinity for the A-T pair;

FIG. 8 shows a graph showing a malaria treatment effect with a PIPA of the present disclosure in humanized mice in one embodiment of the present disclosure; and

FIG. 9 shows a schematic diagram showing a conjugate of the present disclosure and structures of MBX-4055 and its derivatives for the conjugate in one embodiment of the present disclosure.

### DESCRIPTION OF EMBODIMENTS

The present disclosure will be described with reference to the best mode thereof. It should be understood that throughout this specification, singular expressions also include the concept of their plural forms, unless otherwise noted. Thus, it should be understood that singular articles (e.g., “a”, “an”, “the”, etc. in English) also include the concept of their plural forms, unless otherwise noted. It should also be understood that terms used herein are to be used in the sense normally used in the art, unless otherwise noted. Therefore, unless otherwise defined, all technical and scientific terms used herein have the same meaning as commonly understood by those skilled in the art to which this disclosure pertains. In the event of a conflict, this specification (including definitions) shall prevail.

As used herein, the phrase “pseudo-transcription factor” is broadly construed and refers to a substance that has a property of binding to a conserved sequence to which a particular transcription factor specifically binds and that inhibits transcription that is performed by a transcription factor via a binding sequence therefor, and/or activation of the transcription factor.

Definitions and/or basic technical details of terms specifically used herein will be described as appropriate.

As used herein, the term “about” means±10% of the numeral value that follows.

As used herein, the phrase “pyrrole imidazole polyamide (PIPA)” is a low molecular weight organic compound that primarily includes a pyrrole-containing amino acid residue and an imidazole-containing amino acid residue as its building blocks. The PIPA is known to bind to double-stranded DNA more strongly and sequence-specifically than a transcription factor, thereby strongly suppressing a transcriptional activity of a target gene. A surface of a DNA double helix structure has two types of grooves, deep and shallow grooves, and the PIPA enters the shallow groove (minor groove) and reversibly binds to each base of DNA via a hydrogen bond. The PIPA is a condensate of amino acids and can be considered a polypeptide. However, since the PIPA is completely artificial, it is not degraded by various proteolytic enzymes in vivo and is stable in vivo. The PIPA also has an advantage of not requiring a drug delivery system (DDS) because of its property of easily passing through a biomembrane.

As used herein, the term “protozoon” is broadly construed and refers to an organism that infects human and other animals to thereby cause harm to an infected subject. For example, examples of the protozoon include, but are not limited to, infectious agents causing malaria, leishmaniasis, toxoplasmosis, cryptosporidiosis, coccidiosis, babesiosis, theileriosis, cystoisosporiasis, and other protozoan infections.

As used herein, the phrase “protozoan transcription factor” refers to a transcription factor that functions in a protozoan body. In the narrow sense, the protozoan transcription factor refers to a protozoon-specific transcription factor that functions only in a protozoon, and in the broader sense, the protozoan transcription factor also includes a general transcription factor described below.

As used herein, the phrase “general transcription factor” refers to a transcription factor common to all eukaryotes including protozoa. The general transcription factor is a factor required for an RNA polymerase to correctly recognize a promoter and initiate transcription and, for example, there are six general transcription factors that are required for transcription by an RNA polymerase II (TFIIA, TFIIB, TFIID, TFIIE, TFIIF, and TFIIH). Although there are differences among species, all species have proteins similar to them. A sequence recognized by the general transcription factor is generally a sequence containing TATA, called a TATA box, but the general transcription factor may also recognize a unique sequence that is not a TATA box.

As used herein, the phrase “binding region for protozoan transcription factor” refers to a genomic DNA sequence or its region to which a protozoan transcription factor binds.

As used herein, the phrase “specifically bind(s)” means that molecules or substances bind to each other under conditions they do not substantially significantly bind to other molecules or substances. The phrase “specific binding” refers to a binding reaction wherein a PIPA of the present disclosure has a binding affinity of about 500 nM or less as a dissociation constant (Kd value). By “specifically” is preferably meant that a level of binding to molecules other than a target molecule represents a binding affinity of about 50% or less, about 40% or less, about 30% or less, about 20% or less, about 10% or less, more preferably about 5% or less of an affinity for the target molecule. Any method known in the art can be used to measure the dissociation constant, for example, the method exemplified in Example can be used. For a PIPA which specifically binds to a particular binding region, as long as a nucleic acid sequence of a binding region of interest is fixed, a structure of the corresponding PIPA can be identified and this identification can be designed as appropriate with reference to, for example, Journal of the American Chemical Society, 2012, vol. 134, p 17814-17822 by those skilled in the art, of which description is also described elsewhere herein.

As used herein, the term “treatment” means, either prophylactically and/or therapeutically in the broad sense, or for the purpose of amelioration (cure) from a morbid condition in the narrow sense, alleviating, weakening, or ameliorating at least one symptom of a disease or condition, preventing an additional symptom, inhibiting a disease or condition, for example, suppressing an onset of a disease or condition, mitigating a disease or condition, causing regression of a disease or condition, mitigating a condition caused by a disease or condition, or arresting a symptom of a disease or condition. As used herein, the term “treatment” means alleviating, weakening, or ameliorating at least one symptom of a disease or condition for the purpose of amelioration (cure) from a morbid condition.

As used herein, the term “prevention” means preventing a clinical symptom of a disease state from developing in a subject who is at risk of exposure or susceptibility to the disease state, but who has not yet experienced or developed a symptom of the disease state.

As used herein, the term “gene” refers to a factor that determines a genetic trait, and the “gene” may be a nucleic acid itself or may refer to “polynucleotide”, “oligonucleotide”, “RNA”, or “DNA”, and also sometimes refers to a protein, polypeptide, oligopeptide or peptide encoded by a nucleic acid, which can be appropriately understood by those skilled in the art according to the context. A gene encoding such a protein may be endogenous or exogenous to an organism of interest. Such known genes can also be used as appropriate. The gene can be used regardless of an organism from which the gene is derived. That is, the gene may be derived from an organism belonging to a species or genus different from one that an organism of interest belongs to, or may be derived from an organism such as an animal, a plant, a fungus (such as a mold), a bacterium, or the like. Those skilled in the art can obtain information on such a gene by accessing a website such as the NCBI (National Center for Biotechnology Information; http://www.ncbi.nlm.nih.gov) as appropriate. Such a gene may be a gene that encodes a protein having a certain relationship to sequence information disclosed in a database, etc., as long as it exhibits an activity that the protein has.

The terms “protein”, “polypeptide”, “oligopeptide”, and “peptide” are used herein in the same meaning and refer to a polymer of amino acids having any length. The polymer may be linear, branched, or cyclic. The amino acid may be a naturally occurring, non-naturally occurring, or modified amino acid. The terms can also encompass multiple polypeptide chains assembled into a complex. The terms also encompass a polymer of naturally occurring or artificially modified amino acids. Examples of such a modification include, for example, formation of a disulfide bond, glycosylation, lipidation, acetylation, phosphorylation, or any other manipulation or modification (e.g., conjugation with a labeled component). This definition also encompasses, for example, a polypeptide containing one or two or more amino acid analogs (including, for example, non-naturally occurring amino acids), a peptide-like compound (e.g., peptoid), and other modifications known in the art. As used herein, the phrase “amino acid” is a generic term for organic compounds each having an amino group and a carboxyl group. When an antibody according to an embodiment of the present disclosure includes a “particular amino acid sequence”, any of amino acids in the amino acid sequence may be chemically modified. Any of the amino acids in the amino acid sequence may also form a salt or a solvate. In addition, any of the amino acids in the amino acid sequence may be L- or D-type. Even in those cases, a protein according to the embodiment of the present disclosure can be said to include the above “particular amino acid sequence”. Examples of known chemical modifications that an amino acid included in a protein undergoes in vivo include an N-terminal modification (e.g., acetylation, myristoylation, etc.), a C-terminal modification (e.g., amidation, glycosylphosphatidylinositol addition, etc.), or a side chain modification (e.g., phosphorylation, glycosylation, etc.). The amino acid may or may not be naturally occurring as long as it achieves the purpose of the present disclosure.

As used herein, the terms “polynucleotide”, “oligonucleotide”, and “nucleic acid” are used herein in the same meaning, refer to a polymer of nucleotides having any length, and include DNA and RNA. These terms also include “oligonucleotide derivative” or “polynucleotide derivative”. The terms “oligonucleotide derivative” and “polynucleotide derivative” refer to an oligonucleotide or polynucleotide that includes a nucleotide derivative or has an unusual bond between nucleotides, and are used interchangeably. Specific examples of such an oligonucleotide include a 2′-O-methyl-ribonucleotide, an oligonucleotide derivative wherein a phosphodiester bond in an oligonucleotide is converted to a phosphorothioate bond, an oligonucleotide derivative wherein a phosphate diester bond in an oligonucleotide is converted to an N3′-P5′ phosphoramidate bond, an oligonucleotide derivative wherein a ribose-phosphate diester linkage in an oligonucleotide is converted to a peptide-nucleic acid linkage, an oligonucleotide derivative wherein a uracil in an oligonucleotide is replaced with a C-5 propynyl uracil, an oligonucleotide derivative wherein a uracil in an oligonucleotide is replaced with a C-5 thiazole uracil, an oligonucleotide derivative wherein a cytosine in an oligonucleotide is replaced with a C-5 propynyl cytosine, an oligonucleotide derivative wherein a cytosine in an oligonucleotide is replaced with a phenoxazine-modified cytosine, an oligonucleotide derivative wherein a ribose in DNA is replaced with a 2′-O-propylribose, and an oligonucleotide derivative wherein a ribose in an oligonucleotide is replaced with a 2′-methoxyethoxyribose. Unless otherwise indicated, it is contemplated that a particular nucleic acid sequence will also encompass an explicit sequence as well as its conservatively modified modification (e.g., condensed codon substitution) and complementary sequences thereof. Specifically, the condensed codon substitution can be achieved by creating a sequence wherein the third positions of one or more selected (or all) codons are replaced with mixed bases and/or deoxyinosine residues (Batzer et al., Nucleic Acid Res. 19:5081 (1991); Ohtsuka et al. J. Biol. Chem. 260:2605-2608 (1985); Rossolini et al., Mol. Cell. Probes 8:91-98 (1994)). As used herein, the phrase “nucleic acid” is used interchangeably with the terms gene, cDNA, mRNA, oligonucleotide, and polynucleotide. As used herein, the term “nucleotide” may be naturally occurring or non-naturally occurring.

An amino acid may be referred to herein either by its commonly known three-letter code or by one-letter code recommended by the IUPAC-IUB Biochemical Nomenclature Commission. Similarly, a nucleotide can also be referred to by a commonly recognized one-letter code. Comparison of similarity, identity, and homology between amino acid sequences or base sequences is herein calculated using default parameters by BLAST, a tool for sequence analysis. An identity search can be performed, for example, using BLAST 2.2.28 by the NCBI (published Apr. 2, 2013). A value of identity herein usually refers to a value determined as aligned under default conditions using the BLAST. However, if a change in parameters results in a higher value, the highest value shall be a value of identity. When identity is evaluated in a plurality of regions, the highest value shall be a value of identity. Similarity refers to a numerical value calculated taking similar amino acids into account in addition to identity.

### PREFERRED EMBODIMENT

Preferred embodiments of the present disclosure will be described. Embodiments provided below are given for a better understanding of the present disclosure and the scope of the present disclosure should not be limited to the following description. Therefore, it is clear that those skilled in the art may make modifications as appropriate within the scope of the present disclosure, taking into account the description herein. The following embodiments of the present disclosure may also be used alone or in combination.

In an aspect of the present disclosure, a pyrrole imidazole polyamide (PIPA) which specifically binds to a binding region for a protozoa transcription factor is provided.

Parasitic diseases caused by protozoa such as Plasmodium spp. are one of the world's largest infectious disease problems since no radical therapeutic drugs have not yet been developed. In the case of malaria infection, for example, antimalarial drugs such as hydroxychloroquine and artemisinin have been developed, but it remains a serious infectious disease that is far from eradicated due to emergence of drug-resistant protozoa.

For example, Plasmodium spp. have a very unique transcription mechanism that is regulated only by an Apicomplexa AP2 (ApiAP2) family transcription factor (TF). This is an essential TF that is highly conserved among protozoa of the phylum Apicomplexa including Plasmodium spp., Toxoplasma spp., and Cryptosporidium spp., whereas in higher eukaryotes, transcription is regulated via a complex mechanism by various transcription factors. In fact, AP2-O, one of stage-specific AP2 TFs, regulates expression of 500 genes or more, including genes that are essential for proceeding to at least the succeeding form. These facts imply that the AP2 TF could be an attractive target for antimalarial drugs that prevent emergence of drug-resistant mutants.

A pyrrole imidazole polyamide (PIPA) is a low molecular weight organic compound with a sequence-specific DNA binding activity and can directly inhibit transcription of a particular target gene. The PIPA binds to an appropriate region on a promoter, thereby inhibiting a function of a TF and, in turn, transcription of the target gene. The PIPA is efficiently transported into a nucleus both in vitro and in vivo without the use of a drug delivery system (DDS), giving it a significant advantage over other nucleic acid-based gene suppression technologies such as siRNA that cannot penetrate a cell membrane. Although the PIPA can theoretically regulate transcription in all organisms, the PIPA has been used to regulate transcription primarily in mammalian cells and has never been used for protozoa. To date, the PIPA has been used primarily to specifically inhibit one target gene or, rarely, a plurality of genes, and has not been used to function as a pseudo-transcription factor.

In one embodiment of the present disclosure, a PIPA of the present disclosure specifically binds to a binding region for a transcription factor of a protozoon. The protozoon is not particularly limited as long as its morphological change in each life cycle is regulated by a transcription factor, and, for example, may include Plasmodium spp., Leishmania spp., Toxoplasma spp., Cryptosporidium spp., a coccidium., Babesia spp., Theileria spp., and Cystoisospora spp. In one embodiment, the protozoon may be protozoa belonging to the phylum Apicomplexa. In one embodiment, a PIPA of the present disclosure, which can function as a pseudo-transcription factor, can specifically bind to a binding region for an AP2 family transcription factor. In another embodiment, it is understood that a protozoon targeted by the PIPA of the present disclosure may be other than those exemplified above such as Plasmodium spp., Leishmania spp., Toxoplasma spp., Cryptosporidium spp., a coccidium., Babesia spp., Theileria spp., and Cystoisospora spp. as long as its transcription factor is an AP2 family transcription factor.

Plasmodium spp. are transmitted by anopheline, and sporozoites, a salivary gland-infected form, are injected into a host's body and invade liver cells. Then, the sporozoites split into merozoites, which are released into blood and infect red blood cells. The merozoites repeatedly grow and proliferate in the order of ring forms, trophozoites, and schizonts within red blood cells. Some of them take a form called gametocytes, which differentiate into oocysts when they enter the mosquito's body through blood-sucking, and new sporozoites are produced in the oocysts. In one embodiment of the present disclosure, a PIPA of the present disclosure can inhibit any morphological change in each life cycle, in particular, at least a morphological change to a gametocyte. In one embodiment, a PIPA of the present disclosure can inhibit a morphological change to a schizont.

As shown in Examples, the present disclosure reveals for the first time that a PIPA that can function as a pseudo-transcription factor, for example, AP2-PIPA can pass through three membranes, that is, a nuclear membrane of a protozoon, a cell membrane of the protozoon, and a cell membrane of an infected cell (e.g., red blood cell) and inhibit a function of a protozoan transcription factor in a protozoon such as Plasmodium spp. parasitic in a cell, and thus, can be used as a therapeutic or prophylactic agent for a disease caused by a protozoon. The present disclosure also reveals for the first time that a PIPA that can function as a pseudo-transcription factor is effective for a primitive transcriptional regulatory mechanism (transcriptional regulatory mechanism that has only a small number of transcription factors compared to that in mammals, that is, one, or a few, or no transcription factors, or transcriptional regulatory mechanism wherein a particular conserved DNA sequence is commonly important for regulation of a plurality of genes). In one embodiment, a PIPA of the present disclosure that can function as a pseudo-transcription factor can be used to prevent an original transcription factor from binding to a binding sequence therefor, thereby inhibiting transcription associated with the binding and its activity.

Specifically, the present disclosure provides the only method for effectively and specifically inhibiting a transcriptional regulatory mechanism in a protozoon which is intracellularly and extracellularly parasitic and has a primitive transcriptional regulatory mechanism. Therefore, a protozoon wherein a function of a transcription factor is inhibited by a PIPA of the present disclosure may be any protozoon as long as it has a primitive transcriptional regulatory mechanism. Examples of the protozoon having a primitive transcriptional regulatory mechanism may include Leishmania spp., Trypanosoma spp., Entamoeba histolytica, and Trichomonas spp. in addition to those belonging to the phylum Apicomplexa. When targeting a protozoon of which transcription factor has not yet been identified, the effect that a PIPA of the present disclosure exerts can be achieved by targeting a conserved sequence on a promoter revealed by genome analysis, etc.

For a primitive transcriptional regulatory mechanism in a protozoon other than those belonging to the phylum Apicomplexa, for example, in the case of Leishmania spp., it has been known that a long polycistronic mRNA is produced (Journal of Biomedicine and Biotechnology Volume 2010, Article ID 525241, 15 pages). It has been known that a long polycistronic mRNA is produced also in the case of Trypanosoma spp. (Trends in Parasitology October 2011, Vol. 27, No. 10). In the case of Entamoeba histolytica, three consensus sequences have been identified, although the number of transcription factors is unknown (Front. Microbiol. 10:1921. doi: 10.3389/fmicb.2019.01921). In the case of Trichomonas spp., IBP39 has been known to regulate about 75% or more of genes (Molecular Microbiology. 2021; 115:959-967.). Therefore, by designing a PIPA to target a particular sequence or factor that functions in such a primitive transcriptional regulatory mechanism, the PIPA can inhibit a function of the primitive transcriptional regulatory mechanism and be used as a therapeutic or prophylactic agent for a disease caused by a protozoon.

In one embodiment, a PIPA of the present disclosure can specifically bind to a binding region for a protozoon-specific transcription factor. In one embodiment, in the case of a PIPA that targets a general transcription factor, a delivery means such as a protozoon-specific delivery means can specifically inhibit a protozoan transcriptional function.

Dervan et al. demonstrated that a synthetic pyrrole imidazole polyamide (PIPA) has excellent specificity and binds to DNA with very high affinity (Nature 382, 559-561 (1996)). DNA recognition by a PIPA depends on amino acid pairing of an imidazole-pyrrole or pyrrole-pyrrole pair in a minor groove.

The pairing rule for N-methylpyrrole (Py) and N-methylimidazole (Im) amino acid-derived minor groove-bound polyamides determines sequence specificity of a PIPA. Specifically, a Py/Im pair targets a C-G base pair, a Py/Py pair targets A-T and T-A base pairs, and an Im/Py pair targets a G-C base pair.

In one embodiment, a PIPA of the present disclosure binds to dsDNA according to the pairing rule for recognition of a polyamide subunit in a nucleotide base. More specifically, derivatives of pyrrole, imidazole, and 3-hydroxypyrrole, as well as aliphatic amino acid residues arranged in layers form a structure that recognizes a particular target nucleotide base pair in a minor groove of dsDNA. Selected aromatic and aliphatic amino acids are incorporated into polyamide, with the residues remaining unpaired with other amino acid residues. A polyamide molecule is crescent-shaped and can form a complex with a minor groove of double-stranded DNA.

In one embodiment, two polyamides (PIPAs) can be covalently linked by a turn unit such as γ-aminobutyric acid in order to increase a binding affinity to a target sequence. Such polyamides are called “hairpin polyamides” because of their hairpin-like structure in a DNA complex. Sequences of imidazole and pyrrole carboxamides in the polyamide determine DNA sequence specificity of a ligand according to a scheme for a carboxamide to recognize a nucleotide pair. In some cases, one or several pyrrole carboxamide units can be substituted with β-alanine moieties in order to modulate curvature of DNA and a polyamide. A polyamide with chiral R2,4-diaminobutyric acid instead of γ-aminobutyric acid as a turn unit can bind to DNA with a further higher affinity.

In one embodiment, a PIPA of the present disclosure can include an aliphatic amino acid residue in its structure, and the aliphatic amino acid residue can include a molecule having an amino group and a carboxy group. In one embodiment, the aliphatic amino acid residue can include glycine, β-alanine, γ-aminobutyric acid, R2,4-diaminobutyric acid, and 5-aminovaleric acid.

3-hydroxy-N-methylpyrrole (Hp), an aromatic amino acid, can be incorporated into a PIPA and can be designed as a polyamide-DNA binding ligand that is paired with an opposing Py to distinguish an A-T nucleotide pair from a T-A nucleotide pair. Substitution of one hydrogen atom on pyrrole with a hydroxy group in an Hp/Py pairing limits an affinity and specificity of a polyamide by a factor of 10. Use of Hp along with Py and Im in four pairs of aromatic amino acid residues (Im/Py, Py/Im, Hp/Py, and Py/Hp) enables design and synthesis of a polyamide that selectively distinguish all four Watson-Crick base pairs in a minor groove of double-stranded DNA.

In a preferred aspect, the present disclosure provides a PIPA with a carboxamide bond that distinguishes among A-T, T-A, C-G, and G-C base pairs in a minor groove of dsDNA. The present disclosure encompasses a PIPA that has γ-aminobutyric acid and forms a hairpin loop with an element of each carboxamide pair on each end thereof. Preferably, γ-aminobutyric acid is chiral (R)-2,4-diaminobutyric acid.

The present disclosure also encompasses a PIPA including β-alanine substituted for Py which is usually used in a carboxamide bond pair pairing with a particular nucleotide pair. The β-alanine is represented as β in a formula. The β becomes a member of the carboxamide bond pair and acts to optimize a hydrogen bond with a nucleotide pair in an adjacent amino acid moiety. The present disclosure further encompasses substitution of a β-β bond pair with a non-Hp-containing bond pair. Thus, the bond pairs are Py/Py, Im/Py, Py/Im, Im/β, β/Im, Py/β, β/Py, and β/β, in addition to Hp/Py and Py/Hp.

In general, the present disclosure provides a PIPA suitable for inhibiting a morphological change to each life cycle in a protozoon, the PIPA including Py and Im structures selected to correspond to a nucleotide sequence of an identified dsDNA target, an aliphatic amino acid residue selected from the group consisting of glycine, β-alanine, γ-aminobutyric acid, R2,4-diaminobutyric acid, and 5-aminovaleric acid, and optionally a terminal alkylamino residue.

In one embodiment of the present disclosure, a PIPA of the present disclosure may have a hairpin structure or a cyclic structure according to the rules as described above, or two linear PIPAs may be used in combination.

In one embodiment of the present disclosure, a PIPA of the present disclosure includes at least one aliphatic amino acid residue that is β-alanine. In a preferred aspect, the terminal alkylamino residue is an N, N-dimethylaminopropyl residue. A hairpin molecule is formed by an aliphatic amino acid residue, e.g. γ-aminobutyric acid or more preferably R2,4-diaminobutyric acid.

In one embodiment of the present disclosure, a binding region for a protozoan transcription factor to which a PIPA of the present disclosure specifically binds includes 5′-TGCATG-3′ (SEQ ID NO: 1) or an altered sequence thereof, and the altered sequence can include a sequence wherein any one base in 5′-TGCATG-3′ (SEQ ID NO: 1) is deleted or mutated and a sequence wherein one base is added at any position in 5′-TGCATG-3′ (SEQ ID NO: 1). Such a binding region includes NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.

In one embodiment of the present disclosure, a binding region for a protozoan transcription factor to which a PIPA of the present disclosure specifically binds includes 5′-TGCACT-3′ (SEQ ID NO: 8) or an altered sequence thereof and the altered sequence can include a sequence wherein any one base in 5′-TGCACT-3′ (SEQ ID NO: 8) is deleted or mutated and a sequence wherein one base is added at any position in 5′-TGCACT-3′ (SEQ ID NO: 8). Such a binding region includes NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.

In one embodiment of the present disclosure, a PIPA of the present disclosure has the following structure:

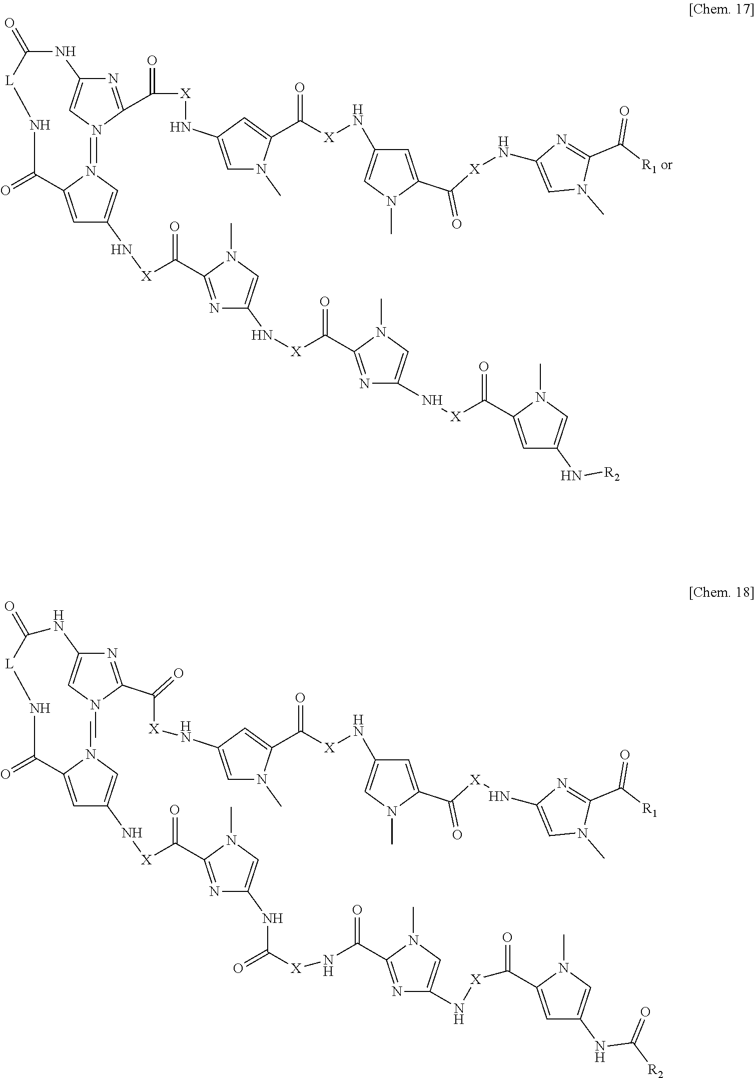

where
L is a C2-6 alkyl linker;
R₁ and R₂ are optionally substituted alkyl, R₁ and R₂ may be taken together with each other to form a C2-6 alkyl linker; and
X may be a bond or an aliphatic amino acid residue.

In one embodiment of the present disclosure, a PIPA of the present disclosure may have the following structure:

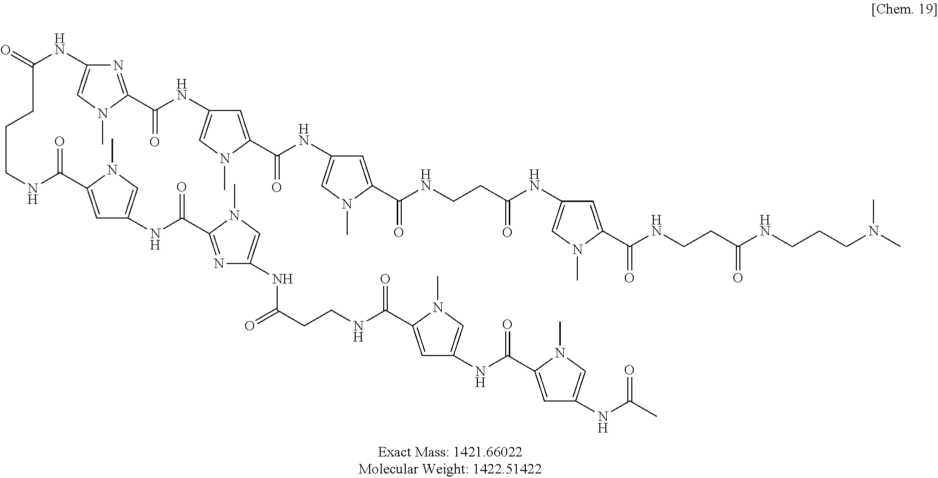 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cc(C(=O)NCCC(=O)NCCCN(C)C)n(C)c8)n(C)c7)n(C)c6)n5)n(C)c4)n3)n(C)c2)n(C)c1]

(AP2-1).

In another embodiment, a PIPA of the present disclosure may have the following structure:

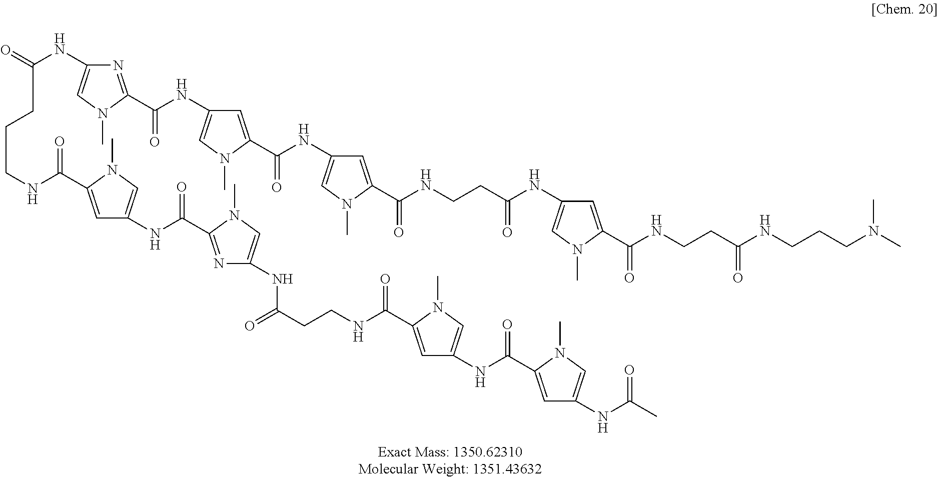 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cc(C(=O)NCCC(=O)NCCCN(C)C)n(C)c8)n(C)c7)n(C)c6)n5)n(C)c4)n3)n(C)c2)n(C)c1]

(AP2-2).

In another embodiment, a PIPA of the present disclosure may have the following structure:

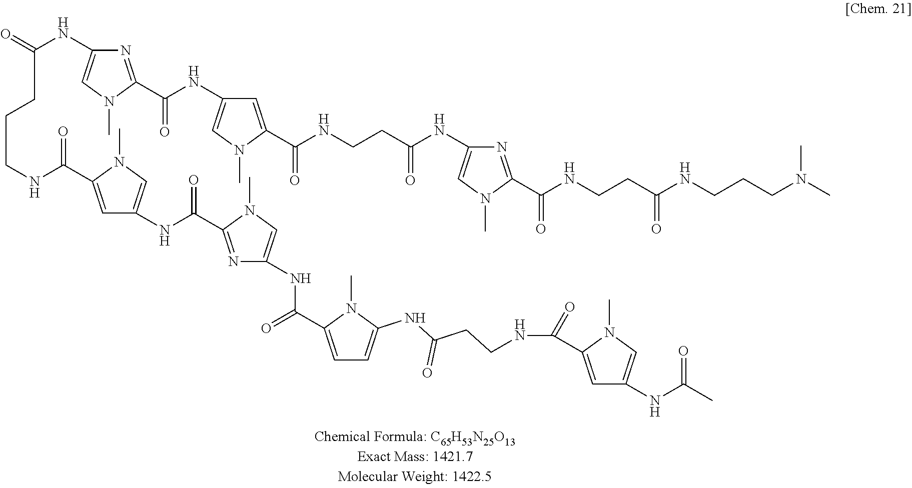 [SMILES: CC(=O)Nc1cc(C(=O)NCCC(=O)Nc2ccc(C(=O)Nc3cn(C)c(C(=O)Nc4cc(C(=O)NCCCC(=O)Nc5cn(C)c(C(=O)Nc6cc(C(=O)NCCC(=O)Nc7cn(C)c(C(=O)NCCC(=O)NCCCN(C)C)n7)n(C)c6)n5)n(C)c4)n3)n2C)n(C)c1]

(AP2-3).

In another embodiment, a PIPA of the present disclosure may have the following structure:

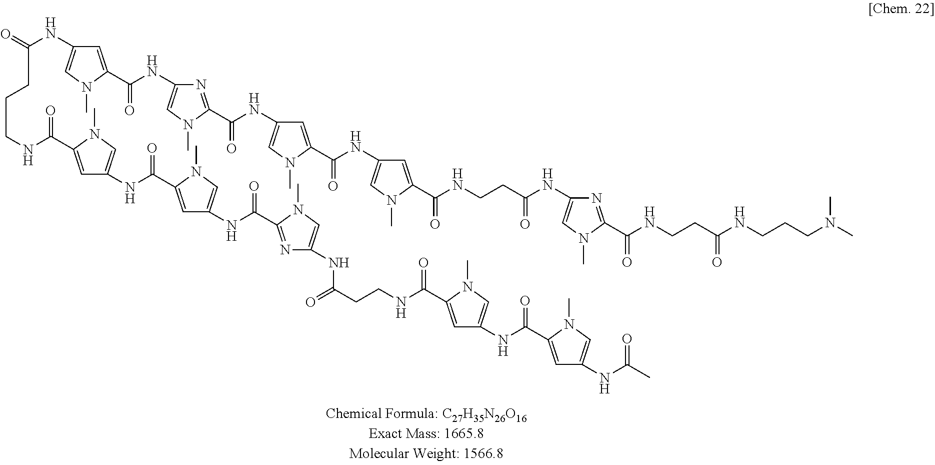 [SMILES: CC(=O)Nc1cc(C(=O)Nc2cc(C(=O)NCCC(=O)Nc3cn(C)c(C(=O)Nc4cc(C(=O)Nc5cc(C(=O)NCCCC(=O)Nc6cc(C(=O)Nc7cn(C)c(C(=O)Nc8cc(C(=O)Nc9cc(C(=O)NCCC(=O)Nc%10cn(C)c(C(=O)NCCC(=O)NCCCN(C)C)n%10)n(C)c9)n(C)c8)n7)n(C)c6)n(C)c5)n(C)c4)n3)n(C)c2)n(C)c1]

(AP2-4).

In another embodiment, a PIPA of the present disclosure may have the following structure:

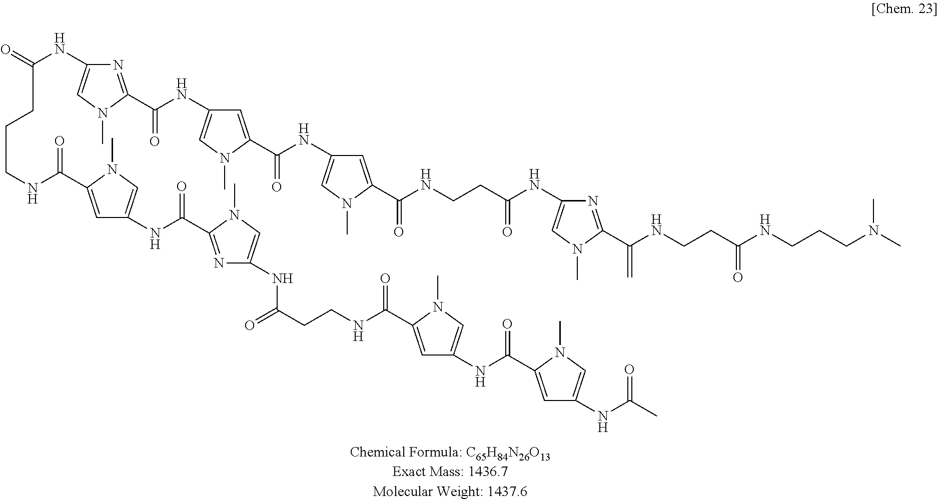 [SMILES: C=C(NCCC(=O)NCCCN(C)C)c1nc(NC(=O)CCNC(=O)c2cc(NC(=O)c3cc(NC(=O)c4nc(NC(=O)CCCNC(=O)c5cc(NC(=O)c6nc(NC(=O)CCNC(=O)c7cc(NC(=O)c8cc(NC(C)=O)cn8C)cn7C)cn6C)cn5C)cn4C)cn3C)cn2C)cn1C]

(AP2-5).

In one embodiment of the present disclosure, a PIPA of the present disclosure can have a binding affinity of about 500 nM or less as a dissociation constant (Kd value) for a binding region. In one embodiment, a PIPA of the present disclosure can have a binding affinity of about 400 nM or less, about 300 nM or less, about 200 nM or less, about 100 nM or less, about 90 nM or less, about 80 nM or less, about 70 nM or less, about 60 nM or less, about 50 nM or less, about 40 nM or less, about 30 nM or less, about 20 nM or less, or about 10 nM or less as a dissociation constant (Kd value) for a binding region. Since a binding strength (dissociation constant) of a transcription factor to a nucleic acid in mammalian cells is approximately 10 nM to several hundred nanomolar, it is assumed that a protozoan transcription factor has a comparable binding strength.

In one embodiment of the present disclosure, a PIPA of the present disclosure is preferably cell permeable and can inhibit gene transcription in vivo, in vitro, or in a cell-free system. Such a polyamide molecule can be appropriately used to inhibit a function of a protozoan transcription factor.

In a related aspect, in one aspect of the present disclosure, there is provided a pyrrole imidazole polyamide (PIPA) which specifically binds to a binding region for a protozoan transcription factor, for inhibiting a function of the protozoan transcription factor.

### (Use as Pseudo-Transcription Factor)

A PIPA of the present disclosure can also be used as a pseudo-transcription factor. Thus, in one aspect of the present disclosure, a pyrrole imidazole polyamide (PIPA) which specifically binds to a binding region for a protozoan transcription factor for use as a pseudo-transcription factor is provided.

In another aspect, a composition including a pyrrole imidazole polyamide (PIPA) which specifically binds to a binding region for a protozoan transcription factor, for use as a pseudo-transcription factor is provided.

In one embodiment, a PIPA of the present disclosure that can function as a pseudo-transcription factor can be used to prevent an original transcription factor from binding to a binding sequence therefor, thereby inhibiting transcription associated with the binding and its activity. Therefore, a function of a protozoan transcription factor can be inhibited by allowing the PIPA of the present disclosure to function as the pseudo-transcription factor. Thus, in one aspect of the present disclosure, a method for inhibiting a function of a protozoan transcription factor in a subject, the method including contacting the protozoan transcription factor in the subject with an effective amount of a pyrrole imidazole polyamide (PIPA) which specifically binds to a binding region for the transcription factor is provided.

In another aspect of the present disclosure, a method for using a pyrrole imidazole polyamide (PIPA) as a pseudo-transcription factor in a subject, the method including applying to the subject an effective amount of a PIPA which specifically binds to a binding region for a protozoan transcription factor is provided.

### (Conjugate)

In another aspect of the present disclosure, a conjugate including a pyrrole imidazole polyamide (PIPA) which specifically binds to a binding region for a protozoan transcription factor and a protozoon-specific factor that is different from the PIPA is provided. A PIPA of the present disclosure can specifically bind to a binding region for a protozoan transcription factor to thereby inhibit its transcription, as described herein. By creating a conjugate of the PIPA with a factor which specifically binds to a protozoon (protozoon-specific factor), a drug delivery system for a PIPA can be constructed.

In one embodiment, a protozoon-specific factor for producing a conjugate of the present disclosure may act directly on a protozoon, or may be a substance that does not act directly but is expected to have some indirect therapeutic effect on the protozoon or suppress and/or prevent a symptom, for example, a substance that activates an immune cell or a substance that protects a red blood cell. A factor that can bind to a PIPA of the present disclosure to thereby constitute a conjugate is preferably of a low molecular weight (about 500 to about 2000), and further preferably of a molecular weight that does not affect a PIPA introduction efficiency, for example, about 500 to about 1000. By constituting a conjugate of such a factor with a PIPA, a conjugate of the present disclosure can provide some therapeutic effect or a symptom suppressive and/or prophylactic effect directly or indirectly on protozoan infection. In one embodiment, a protozoon-specific factor for producing a conjugate of the present disclosure may be those which specifically bind to a protozoon of interest, preferably those which specifically bind to a protozoon of interest to function as an inhibitor. For example, the protozoon-specific factor may include a factor that binds to a surface protein of a malaria-infected red blood cell. Examples of such a factor include a pyridazinone derivative including MBX-4055 and its derivatives represented by the following formulae.

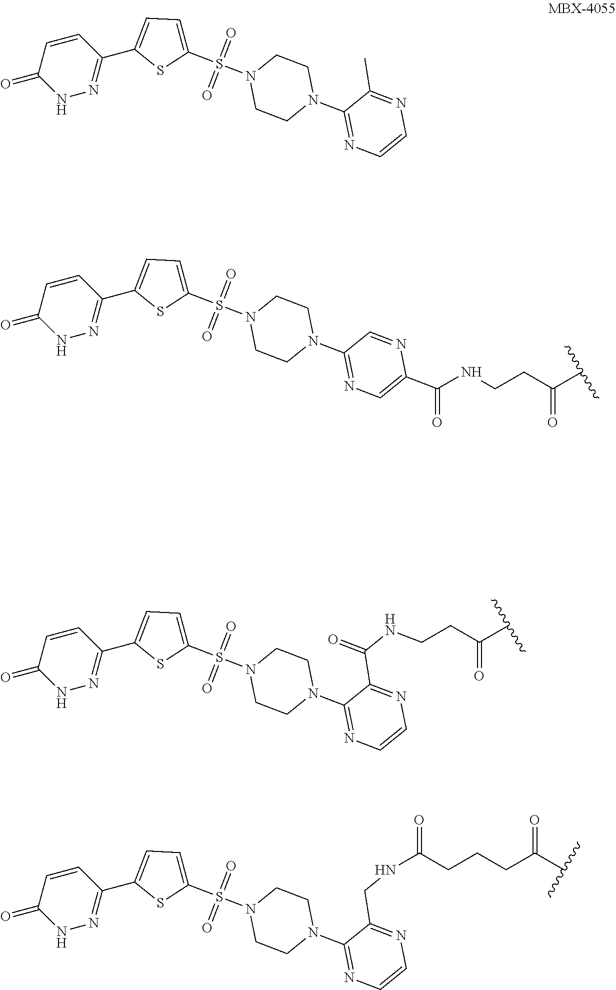 [SMILES: Cc1nccnc1N1CCN(S(=O)(=O)c2ccc(-c3ccc(=O)[nH]n3)s2)CC1 ; *C(=O)CCNC(=O)c1cnc(N2CCN(S(=O)(=O)c3ccc(-c4ccc(=O)[nH]n4)s3)CC2)cn1 ; *C(=O)CCNC(=O)c1nccnc1N1CCN(S(=O)(=O)c2ccc(-c3ccc(=O)[nH]n3)s2)CC1 ; *C(=O)CCCC(=O)NCc1nccnc1N1CCN(S(=O)(=O)c2ccc(-c3ccc(=O)[nH]n3)s2)CC1]

MBX-4055 is known to inhibit proliferation of Plasmodium spp. Specifically, a plasmodial protein expressed on a surface of a red blood cell is required for uptake of a component necessary for plasmodial proliferation from outside the red blood cell, and MBX-4055 inhibits plasmodial proliferation by inhibiting this function. In one embodiment of the present disclosure, MBX-4055 can be used as a drug delivery system, focusing on its action of binding to a plasmodial protein expressed on an infected red blood cell, and can be expected to exert a synergistic effect with the inhibitory effect of a PIPA.

In another embodiment, a protozoon-specific factor may include a factor having a proliferation inhibitory effect on Plasmodium spp., Leishmania spp., Toxoplasma spp., Cryptosporidium spp., and/or a coccidium. Such a factor may include, for example, amphotericin B, a factor that binds to a surface protein of a leishmania-infected cell (Life Sciences, Volume 322, 1 Jun. 2023, 121314). In another embodiment, a protozoon-specific factor may include an inhibitor against Toxoplasma spp., for example, compounds described in Microorganisms 2021, 9 (9), 1960 can be utilized. In another embodiment, a protozoon-specific factor may include an inhibitor against Cryptosporidium spp., for example, compounds described in Animal Diseases, volume 1, Article number: 3 (2021) can be utilized. In another embodiment, a protozoon-specific factor may include an inhibitor against a coccidium, for example, compounds described in J. Jpn. Vet. Med. Assoc 71 166-169 (2018) can be utilized.

In one embodiment, a PIPA of the present disclosure and a protozoon-specific factor may be linked directly to form a conjugate or linked via a linker. Such a linker may be any linker as long as it can exert its function as a conjugate of the present disclosure, e.g., as a drug delivery system, and can include, for example, a C1-6 alkyl linker.

In another aspect, a composition including a pyrrole imidazole polyamide (PIPA) which specifically binds to a binding region for a protozoan transcription factor, for inhibiting a function of the protozoan transcription factor is provided.

In yet another aspect, a protozoan transcription factor inhibitor including a pyrrole imidazole polyamide (PIPA) which specifically binds to a binding region for a protozoan transcription factor is provided.

In one aspect of the present disclosure, a therapeutic or prophylactic agent for a disease caused by a protozoon, the agent including a protozoan transcription factor including a pyrrole imidazole polyamide (PIPA) which specifically binds to a binding region for a protozoan transcription factor is provided.

In one aspect of the present disclosure, a pyrrole imidazole polyamide (PIPA) which specifically binds to a binding region for a protozoan transcription factor, for treating or preventing a disease caused by a protozoon is provided.

In one aspect of the present disclosure, a method for producing a protozoan transcription factor inhibitor including a pyrrole imidazole polyamide (PIPA), the method including providing a binding region for a protozoan transcription factor; and designing the PIPA to specifically bind to the binding region is provided. In one embodiment, the PIPA and the protozoon may be those described elsewhere herein.

In one embodiment, the designing may include linking a pyrrole and/or an imidazole selected to correspond to a nucleotide sequence of the binding region and, if necessary, replacing one or more pyrroles or imidazoles in the linked pyrrole and/or imidazole molecule with β-alanine. In this case, the PIPA is preferably designed and synthesized according to the rule for a PIPA structure as described above.

In one aspect of the present disclosure, a method for treating or preventing a disease caused by protozoon in a subject, the method including administering to the subject an effective amount of a protozoan transcription factor inhibitor, the protozoan transcription factor inhibitor including a pyrrole imidazole polyamide (PIPA) which specifically binds to a binding region for a protozoan transcription factor is provided. In one embodiment, the PIPA and the protozoon may be those described elsewhere herein.

A PIPA of the present disclosure and a pharmaceutically acceptable salt thereof can be formulated into a pharmaceutical or therapeutic composition, formulation, or preparation. The pharmaceutically acceptable salt of the PIPA of the present disclosure is formed with a strong or moderate, non-toxic, organic or inorganic acid or base as appropriate by a method known in the art. Examples of the salt to be included in the present disclosure include a maleate, a fumarate, a lactate, an oxalate, a methanesulfonate, an ethanesulfonate, a benzenesulfonate, a tartrate, a citrate, a hydrochloride, a hydrobromide, a sulfate, a phosphate, and a nitrate.

A PIPA of the present disclosure has an ability to treat or prevent a disease caused by protozoa. A composition of the present disclosure may be active itself or may act as a prodrug that is converted to its active form in vivo.

A PIPA of the present invention and a pharmaceutically acceptable salt thereof can be incorporated into an ordinary dosage form such as a capsule, a tablet, or an injectable preparation. A solid or liquid pharmaceutically acceptable carrier can be used. A pharmaceutical composition designed for delayed release can also be formulated.

Preferably, a PIPA of the present disclosure is administered systemically, e.g., by injection. In use, the injection can be by any known route, preferably intravenously, subcutaneously, intramuscularly, intracranially, or intraperitoneally. An injectable preparation can be prepared in a known form such as either a solution or a suspension, a solid form suitable for dissolving or suspending in liquid before injection, or an emulsion.

A pharmaceutical preparation is prepared according to a conventional technique of medicinal chemistry, including a step of mixing, granulation, and compaction if necessary for a tablet form, or mixing, filling, and dissolving a component if appropriate, thereby obtaining a desired product for oral or parenteral administration including topical, transdermal, vaginal, intranasal, intrabronchial, intracranial, intraocular, intraaural, and rectal administration. A pharmaceutical composition may also include a small amount of a non-toxic auxiliary substance such as a wetting agent or an emulsifying agent and a pH buffering agent.

A preferred route of administration is systemic, but a pharmaceutical composition may also be administered topically or intradermally (e.g., as an ointment, cream, or gel), orally, rectally (e.g., as a suppository), parenterally, continuously by injection or infusion, vaginally, intranasally, intrabronchially, intracranially, intraaurally, or intraocularly. In one embodiment, in the case of a PIPA that targets a general transcription factor, a protozoan transcriptional function can be specifically inhibited by a delivery means such as a protozoon-specific delivery means.

A composition including a PIPA of the present disclosure may also be administered in combination with one or more further compounds to be used to treat a disease or symptom.

An effective amount of a PIPA for treating a disease or symptom can be determined using an in vitro system or an in vivo animal model accepted for a particular disease or symptom.

A treatment method of the present disclosure includes administering an effective amount of a protozoan transcription factor inhibitor. A preparation or formulation containing a PIPA of the present disclosure can be administered systemically or topically and can be used alone or as a mixture of components. A route of administration may be topical, intravenous, oral, or by using an implant. For example, a PIPA can be administered by a means including, but not limited to, a topical preparation, intravenous injection or infusion, oral ingestion, or topical administration in a form of intradermal injection or an implant. A further route of administration is a subcutaneous, intramuscular, or intraperitoneal injection of a PIPA of the present disclosure in a conventional or convenient form. A liposome or a lipophilic formulation can also be used, if desired. For topical administration, a polyamide can be formulated into a standard topical formulation and composition, including a lotion, a suspension, or a paste. If a PIPA can be readily applied to a target cell or tissue by an oral route, oral administration of an appropriate formulation io also suitable.

Those skilled in the art can optimize a dose of a PIPA depending on a factor such as, but not limited to, a PIPA selected, a physical delivery system by which a PIPA is delivered, a patient, and judgment of a skilled physician.

### (General Technologies)

Molecular biological, biochemical, and microbiological procedures used herein are well known and common in the art, and are described in, for example, Sambrook J. et al. (1989). Molecular Cloning: A Laboratory Manual, Cold Spring Harbor and its 3rd Ed. (2001); Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999).

PCR Applications: Protocols for Functional Genomics, Academic Press, and Bessatsu Jikken Igaku “Experimental Methods for Gene Transfer & Expression Analysis”, Yodosha, 1997, etc., each of which is incorporated herein by reference in its relevant parts (may be entirety).

For a DNA synthesis technique and nucleic acid chemistry to produce an artificially synthesized gene, gene synthesis and fragment synthesis services such as GeneArt, GenScript, Integrated DNA Technologies (IDT), etc. can be used and described in, for example, Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approach, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press, etc., each of which is incorporated herein by reference in its relevant parts.

Other references related to a PIPA include Jordan L. Meier et al., J. Am. Chem. Soc. 2012, 134, 17814-17822, Journal of the American Chemical Society, 2012, vol. 134, p 17814-17822, Nature, 1998, vol. 391, p 468, and Advanced Drug Delivery Reviews, 2019, vol. 147, p 66-85, Bull. Chem. Soc., 2020, vol. 93, p 205-215.

As used herein, the term “or” is used when “at least one or more” of matters enumerated in a text can be employed. The same is true for “alternatively”. When the phrase “within a range” of “two values” is specified herein, the range includes the two values themselves.

References cited herein such as scientific literature, patents, and patent applications are incorporated herein by reference in its entirety as if specifically set forth herein.

The present disclosure has been described with reference to preferred embodiments for ease of understanding. Hereinafter, the present disclosure will be described with reference to Examples, but the above description and Examples below are provided for illustrative purposes only and are not intended to limit the present disclosure. Accordingly, the scope of the present disclosure is not limited to embodiments or Examples specifically described herein, but is limited only by claims.

### EXAMPLE

### Reagent

The pyrrole imidazole polyamide was synthesized at PEPTIDE INSTITUTE, INC. (Osaka, Japan). The lipopolysaccharide was purchased from Invivogen (Carlsbad, CA, USA; tlrl-eblps).

### Cell

Raw246.7 cells, NIH3T3 cells, U937 cells, and A549 cells were obtained from ATCC. Red blood cells were obtained from whole blood as described below. Human blood was kindly provided by healthy human volunteers according to the guidelines of the University of Tokyo or Osaka University.

qRT-PCR Analysis

Total RNA used for in vitro stimulation or in vivo injection was extracted from cells using NucleoSpin RNA (TAKARA BIO, Shiga, Japan).

DNA primers were obtained from FASMAC (Kanagawa, Japan).

### Example 1: Transcriptional Inhibitor Against P. falciparum

This example shows an example of a transcriptional inhibitor against P. falciparum.

In order to develop a transcriptional inhibitor against P. falciparum, a target transcription factor and a sequence thereof were first determined. Since one of the most prominent toxic effects of Plasmodium spp. is destruction of red blood cells, we decided to inhibit formation of merozoites, which destroy cells in a final blood stage and cause invasion to new red blood cells. Plasmodium spp. form schizonts just before forming merozoites. In the schizonts, AP2-Sc is expressed and plays an important role as an essential transcription factor.

Therefore, the AP2-Sc was selected as a target, and then ChIP-seq analysis was performed on this target to comprehensively determine binding sequences therefor. As a result, the sequence TGCATG (SEQ ID NO: 1) was the first hit, and about 50% of precipitated DNA segments contained this sequence. The TGCATG (SEQ ID NO: 1) was found to be specific to a promoter region on a schizont-specific gene such as AMA1 (PF3D7 1133400) and GAMA (PF3D7 0828800). On the other hand, other growth stage-specific genes such as CTRP were not found. These results indicate that the TGCATG (SEQ ID NO: 1) may be important in schizont formation in P. falciparum.

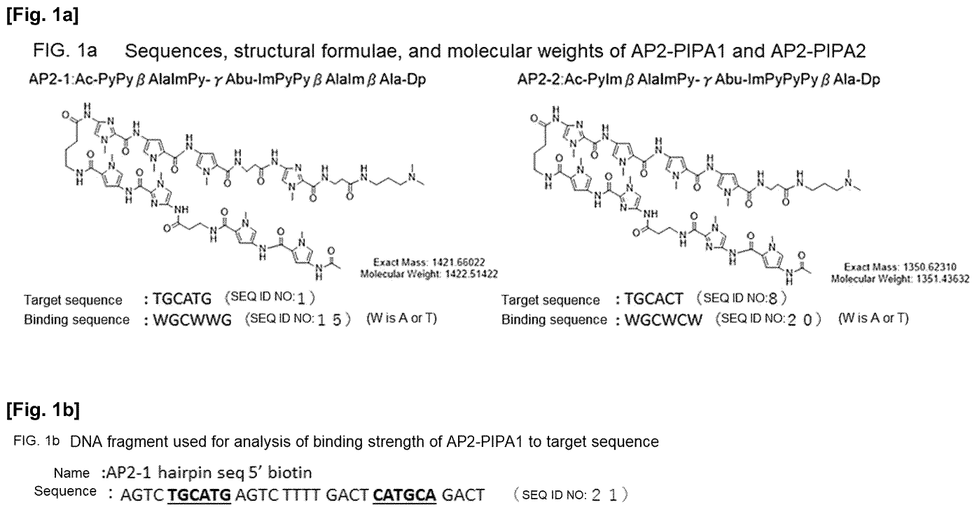

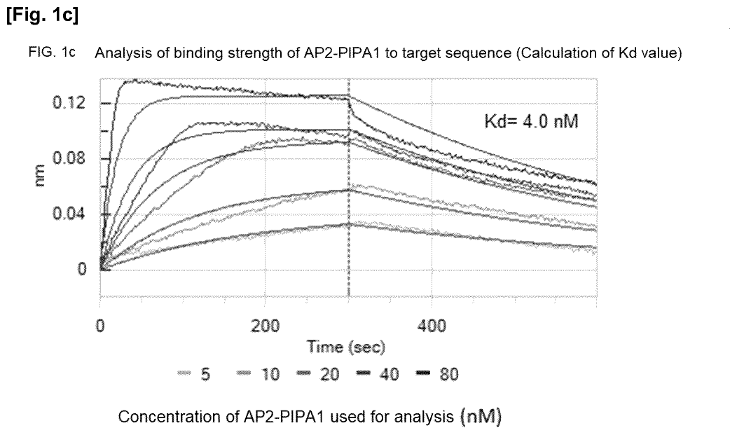

Next, based on the ChIPC-seq analysis, a PIPA targeting the TGCATG (SEQ ID NO: 1) (AP2-PIPA1) and the second hit sequence motif TGCACT (SEQ ID NO: 8) (AP2-PIPA2) were produced as controls. As shown in FIG. 1a, the AP2-PIPA is a hairpin PIPA containing β-alanine in its structure. Total molecular weights were 1423 and 1351, respectively.

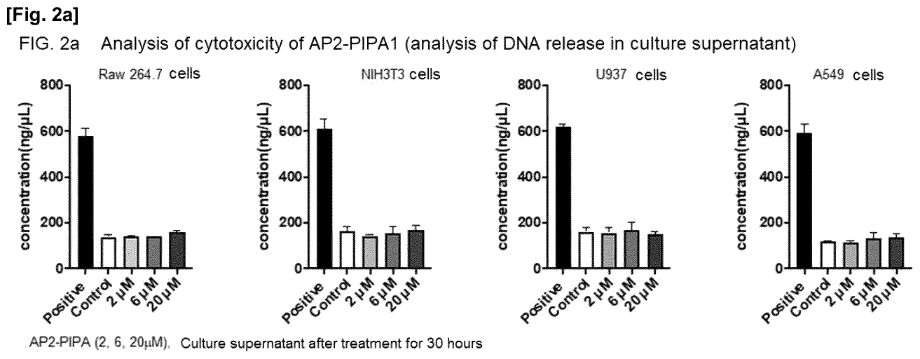

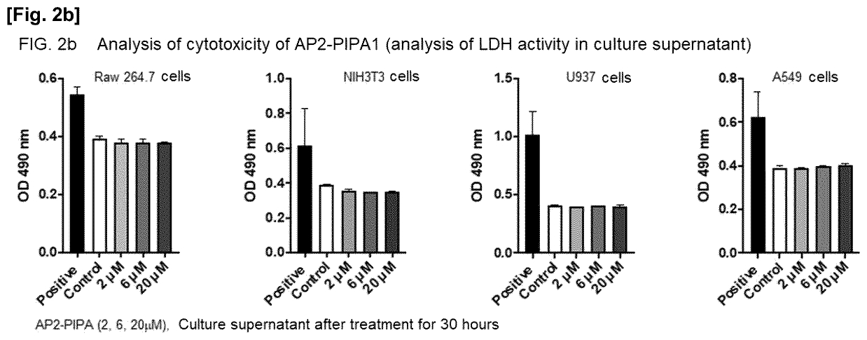

Next, a binding affinity of a PIPA to dsDNA containing the target sequence for the AP2-PIPA (FIG. 1b) was verified. As designed, the AP2-PIPA1 was bound to the target sequence with a high binding activity, that is, a Kd value of 4.0 nM (FIG. 1c). Consistent with previous reports of low off-target toxicity of a PIPA, the AP2-PIPA was not toxic in either mouse cell lines (Raw264.7 cells, NIH3T3 cells) or human cell lines (U937 cells, A549 cells) (FIG. 2a and FIG. 2b). These analysis results support safety of a PIPA at least in the dose range in vitro.

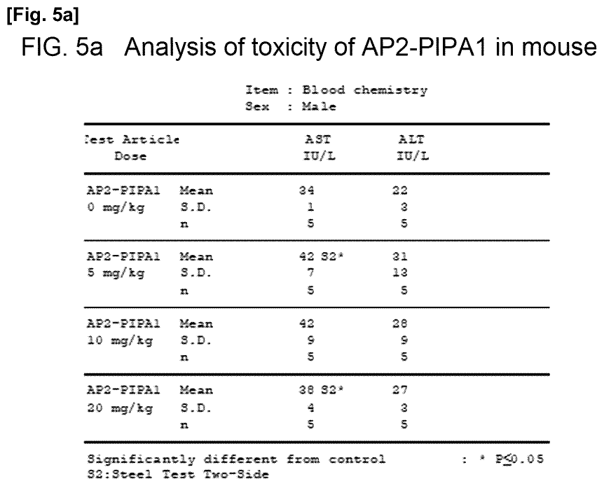

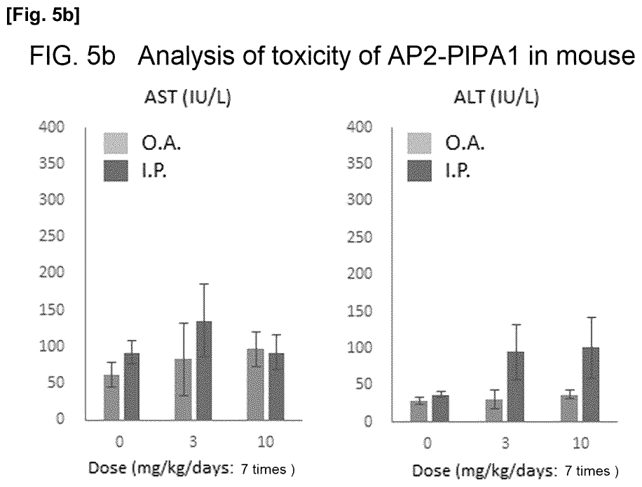

Furthermore, toxicity of a single intraperitoneal administration of the AP2-PIPA1 was studied in mice. Doses were 0 (PBS), 5, 10, and 20 mg/kg, and the mice were observed for 7 days after the administration. Observation of viability and general conditions, measurement of body weight, a hematology test, a blood chemistry test, and necropsy were performed as toxicity assessment indices. No deaths were observed, and no changes that were considered to be abnormal were found in the observation of general conditions, a weight change, the hematology test, the blood chemistry test, or the autopsy. Significant increases in AST activity were seen in the 5 and 20 mg/kg dose groups. For the 10 mg/kg dose group, although the difference was not significant, an upward trend was observed (FIG. 5a). However, the value for each individual in these groups falls within a background value (34 to 58 IU/L) and thus the changes are determined to be due to physiological variation. Based on these results, the minimum lethal dose of the AP2-PIPA1 for the toxicity of a single intraperitoneal administration was estimated to be greater than 20 mg/kg.

Furthermore, toxicity of multiple administrations of the AP2-PIPA1 was studied in mice. Mice received various doses of the AP2-PIPA1 once daily for 7 days orally or intraperitoneally. On day 8, blood was collected and AST and ALT were measured (FIG. 5b). Significant difference tests were performed for the 3 mg/kg and 10 mg/kg dose groups relative to the control group for each route of administration. As a result of the Bartlett's test for Equality of variances, p>0.01 was determined to be equal variance, and as a result of Dunnett's two-tailed test, there was no significant difference at p>0.05. These results suggest that the AP2-PIPA1 is not prominently toxic in the dose range examined in this study.

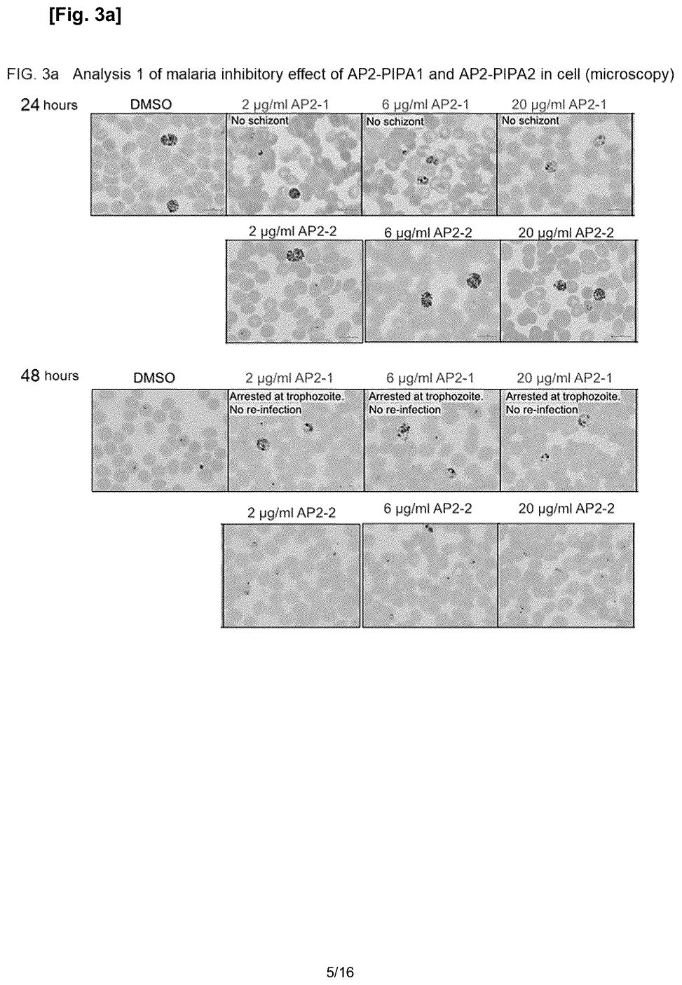

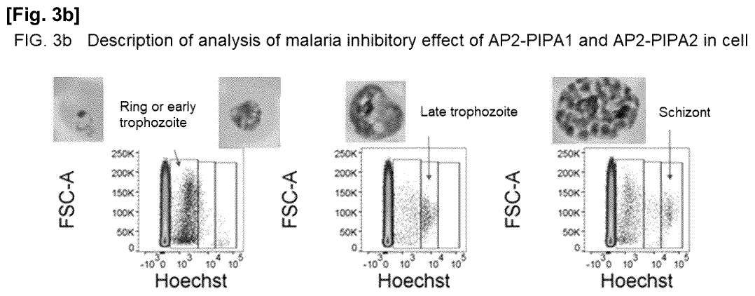

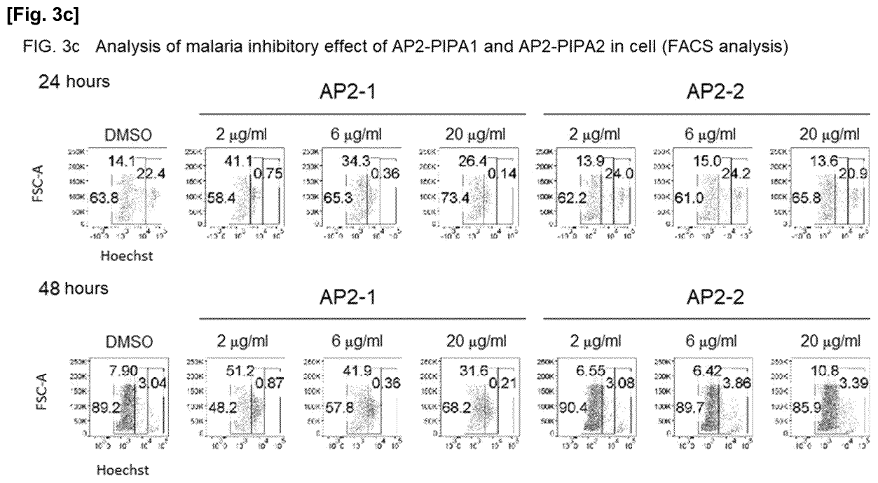

Then, an effect of the AP2-PIPA on proliferation of a P. falciparum 3D7 laboratory strain at a blood stage in vitro was examined. Human red blood cells were infected with P. falciparum and cultured in the presence or absence of the AP2-PIPA. Microscopic observation with Giemsa staining and quantitative count of parasites and their stages by FACS analysis were performed at 24 and 48 hours after the infection. As shown in FIG. 3a, the AP2-PIPA1 arrested a life cycle of P. falciparum at a trophozoite stage in a more dose-dependent manner than the AP2-PIPA2. As a result of the FACS analysis, for P. falciparum cultured in the presence of the AP2-PIPA1, a morphological change was arrested at the trophozoite stage at both 24 and 48 hours after the infection (FIGS. 3b and 3c). These results suggest that the AP2-PIPA1 does not affect trophozoite formation but specifically inhibits transition to a schizont stage, consistent with a role for the target sequence binding to the AP2-Sc. The AP2-PIPA2 was thought to be effective at a higher concentration range than that used in this experiment.

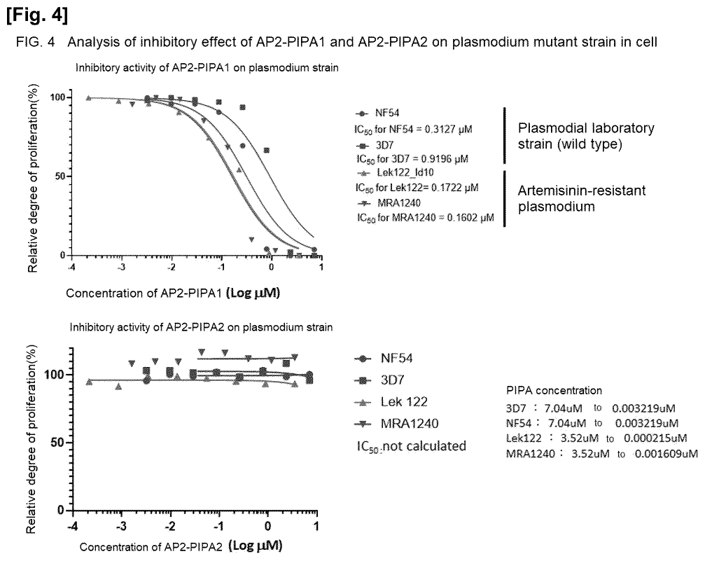

An effect of the AP2-PIPA on proliferation of an artemisinin-resistant strain in vitro was further examined (FIG. 4). Human red blood cells were infected with various Plasmodium strains and cultured in the presence or absence of the AP2-PIPA at various concentrations. Parasitemia was evaluated at 72 hours after the infection to verify a degree of proliferation of Plasmodium treated with various concentrations of the AP2-PIPA, and an IC₅₀ value for the AP2-PIPA in each Plasmodium strain was calculated from the above results. As a result, the AP2-PIPA1 inhibited proliferation of the artemisinin-resistant plasmodium strain with a lower IC₅₀. On the other hand, the AP2-PIPA2 showed no inhibitory effects on any of the Plasmodium strains, suggesting that it is effective at a higher concentration range than that used in this experiment.

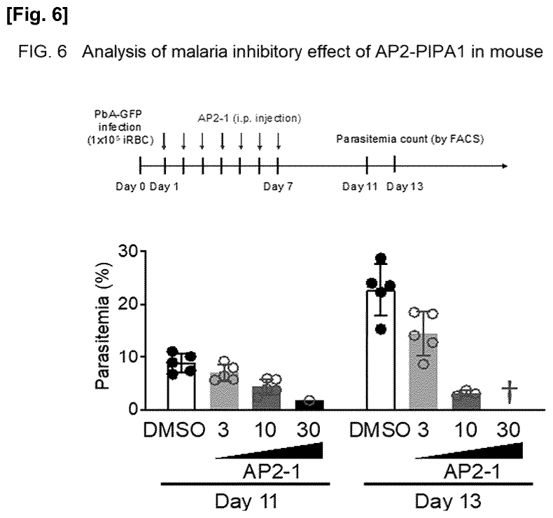

Furthermore, an effect of the AP2-PIPA1 was verified in a mouse infection experiment. Mice were infected with a Plasmodium berghei strain and received the AP2-PIPA1 intraperitoneally at each time point (day 1, day 2, day 3, day 4, day 5, day 6, and day 7) shown in the upper schematic diagram in FIG. 6. Blood was collected at 11 or 13 days after the infection to assess parasitemia. As a result, a dose-dependent suppression of parasitemia was observed. For higher dose groups, particularly the 30 mg/kg dose group, suppression of parasitemia and mouse death were observed (see the lower graph in FIG. 6). Based on these results, an antimalarial effect of the AP2-PIPA1 in mice was confirmed. Multiple administrations of a high dose, i.e., 30 mg/kg are believed to cause toxicity.

In this example, we were able to develop a new PIPA-based inhibitor that targets a consensus binding sequence of AP2-Sc, an AP2 family TF that is important for P. falciparum to become schizonts. Unlike the conventional strategy of determining a target sequence of a PIPA based on promoter analysis information, an AP2 TF binds to cis-elements of multiple target genes, so we focused on binding sequences for the AP2-SC revealed by Chromatin Immunoprecipitation sequencing (ChIP-seq) analysis. This strategy allows an AP2-Sc-targeting PIPA (AP2-PIPA) to compete with the AP2-SC not only for a particular gene, but also for all genes with a consensus cis-element on a promoter. In fact, the AP2-PIPA was found to strongly inhibit schizont formation by P. falciparum in an in vitro culture. This is the first time that a PIPA has been shown to inhibit proliferation and a life cycle of a protozoon.

Although the AP2-PIPA can compete with the AP2-Sc for all genes with a consensus cis-element on a promoter, examples of the genes include ARNP (PF3D7 0511600), MSP7 (PF3D7 1335100), MSP9 (1228600), and EXP1 (PF3D7 1121600).

### Example 2: Inhibition of Transcription Factor Using PIPA in Leishmania Spp

It has been known that a long polycistronic mRNA is produced as a primitive transcriptional regulatory mechanism in Leishmania spp. (Journal of Biomedicine and Biotechnology Volume

2010, Article ID 525241, 15 pages). Specifically, a plurality of proteins are produced from this particular mRNA, so a PIPA that inhibits transcription of this polycistronic mRNA was designed. The design methodology is the same as in Example 1.

A binding affinity of a PIPA to a target sequence is examined and a PIPA that was confirmed to bind to the target sequence with a high binding activity is administered to a subject in the same manner as in Example 1. An inhibitory effect of a transcription function in Leishmania spp. was checked. Inhibitory effect and mouse infection experiments are performed in the same manner as in Example 1. Thus, a PIPA that has a therapeutic effect on leishmania infection and its concentration are found.

### Example 3: Inhibition of Transcription Factor Using PIPA in Toxoplasma Spp

The AP2-PIPA1 is thought to inhibit Toxoplasma spp., which belongs to the same phylum Apicomplexa as Plasmodium spp. The rationale is, for example, that an AP2 transcription factor is a transcription factor conserved across the phylum Apicomplexa and that a DNA region to which the AP2 transcription factor binds is conserved, that is, a DNA sequence to be bound is thought to be conserved. In fact, as shown in FIG. 4 of THE JOURNAL OF BIOLOGICAL CHEMISTRY VOL. 288, NO. 43, pp. 31127-31138 and FIG. 1 of PNAS Jun. 17, 2008, vol. 105, no. 248393, TgAP2XI-5, one of the AP2 transcription factors of Toxoplasma spp., is an important transcription factor involved in expression of 300 genes or more and its AP2-DNA binding domain shows high homology to that for an AP2 transcription factor of Plasmodium spp. Furthermore, as shown in FIG. 4 and FIG. 6 in the above literature, the most probable DNA sequence to which the TgAP2XI-5 binds is expected to be GCTAGC, which is different from a sequence that can be inhibited by the AP2-PIPA1 (WGCWWG (SEQ ID NO: 15) (W may be either A or T) by only one base. Correctly, if a nucleotide on the immediate 5′ side of GCTAGC (SEQ ID NO: 16) is A or T, they are perfectly matched. Specifically, there is a 50% chance of perfect match. Even in the case of the remaining 50% of mismatches, the AP2-PIPA1 is believed to bind, although a binding strength may be reduced due to a single nucleotide mismatch. Furthermore, as shown in FIG. 4 and FIG. 6 in the above literature, the DNA sequence (agctag) (SEQ ID NO: 17) that is the fourth most probable sequence bound by the TgAP2XI-5 in FIG. 4 and the third most probable sequence bound by the TgAP2XI-5 in FIG. 6 perfectly matches the sequence (WGCWWG) that can be inhibited by the AP2-PIPA1 (SEQ ID NO: 15). In other literatures, one of the most probable sequences recognized by AP2 transcription factors in the phylum Apicomplexa including Plasmodium spp., Toxoplasma spp., Cryptosporidium spp., and a coccidium is reported as TGCATGCA (SEQ ID NO: 18) (e.g., Pathogens 2019, 8, 47; doi: 10.3390/pathogens8020047) (Genome Res. 2007 17:311-319), which includes a sequence that is 100% identical to the sequence (WGCWWG (SEQ ID NO: 15) (W may be either A or T) that can be inhibited by the AP2-PIPA1.

Based on the above, AP2-PIPA1, which was designed in the same manner as in Example 1, is used to verify an inhibitory effect of a transcription factor in Toxoplasma spp. Inhibitory effect and mouse infection experiments are performed in the same manner as in Example 1. Thus, a concentration of the AP2-PIPA1 that has a therapeutic effect on toxoplasma infection is determined.

### Example 4: Inhibition of Transcription Factor Using PIPA in Cryptosporidium Spp

The AP2-PIPA1 is expected to inhibit Cryptosporidium spp., which belongs to the same phylum Apicomplexa as Plasmodium spp. The rationale is, for example, that an AP2 transcription factor is a transcription factor conserved across the phylum Apicomplexa and that a DNA region to which the AP2 transcription factor binds is conserved, that is, a DNA sequence to be bound is thought to be conserved. In fact, as shown in FIG. 1 in PNAS Jun. 17, 2008, vol. 105, no. 248393, an AP2-DNA binding domain for a cryptosporidial AP2 shows high homology to that for an AP2 transcription factor of Plasmodium spp. Furthermore, DNA sequences bound by a cryptosporidial AP2 shown by Cad8_3230, Cgd1_3520, and Cgd2_3490 in FIG. 3 of Nucleic Acids Research, 2014, Vol. 42, No. 13 includes a sequence that perfectly matches the sequence (WGCWWG (SEQ ID NO: 15) (W may be either A or T) that can be inhibited by the AP2-PIPA1. Furthermore, the most probable DNA sequence bound by a cryptosporidial AP2 shown in FIG. 5 of the same literature includes a sequence that perfectly matches the sequence that can be inhibited by the AP2-PIPA1 (WGCWWG (SEQ ID NO: 15) (W may be either A or T). Similar facts are also presented in PNAS Jun. 17, 2008, vol. 105, no. 248393. In other literature, one of the most probable sequences recognized by AP2 transcription factors in the phylum Apicomplexa including Plasmodium spp., Toxoplasma spp., Cryptosporidium spp., and a coccidium is reported as TGCATGCA (SEQ ID NO: 18) (Pathogens 2019, 8, 47; doi: 10.3390/pathogens8020047) (Genome Res. 2007 17:311-319), which includes a sequence that is 100% identical to the sequence (WGCWWG (SEQ ID NO: 15) (W may be either A or T) that can be inhibited by the AP2-PIPA1.

Based on the above, AP2-PIPA1, which was designed in the same manner as in Example 1, is used to verify an inhibitory effect of a transcription factor in Cryptosporidium spp. Inhibitory effect and mouse infection experiments are performed in the same manner as in Example 1. Thus, a concentration of the AP2-PIPA1 that has a therapeutic effect on cryptosporidium infection is determined.

### Example 5: Inhibition of Transcription Factor Using PIPA in Coccidium

The AP2-PIPA1 is expected to inhibit a coccidium, which belongs to the same phylum Apicomplexa as Plasmodium spp. The rationale is, for example, that an AP2 transcription factor is a transcription factor conserved across the phylum Apicomplexa and that a DNA region to which the AP2 transcription factor binds is conserved, that is, a DNA sequence to be bound is thought to be conserved. Since it has been shown that all of AP2 transcription factors of Plasmodium spp., Toxoplasma spp., and Cryptosporidium spp., which belong to the same phylum Apicomplexa, bind to the sequence that can be inhibited by the AP2-PIPA1 (WGCWWG (SEQ ID NO: 15) (W may be either A or T), a coccidial AP2 is also thought to bind to a sequence that can be inhibited by the AP2-PIPA1. In fact, one of the most probable sequences recognized by AP2 transcription factors in the phylum Apicomplexa including Plasmodium spp., Toxoplasma spp., Cryptosporidium spp., and a coccidium is reported as TGCATGCA (SEQ ID NO: 18) (Pathogens 2019, 8, 47; doi: 10.3390/pathogens8020047) (Genome Res. 2007 17:311-319), which includes a sequence that is 100% identical to the sequence (WGCWWG (SEQ ID NO: 15) (W may be either A or T) that can be inhibited by the AP2-PIPA1.

Based on the above, AP2-PIPA1, which was designed in the same manner as in Example 1, is used to verify an inhibitory effect of a transcription factor in a coccidium. Inhibitory effect and mouse infection experiments are performed in the same manner as in Example 1. Thus, a concentration of the AP2-PIPA1 that has a therapeutic effect on coccidium infection is determined.

### Example 6: Inhibition of Transcription Factor Using PIPA in Babesia Spp

The AP2-PIPA1 is expected to inhibit Babesia spp., which belongs to the same phylum Apicomplexa as Plasmodium spp. The rationale is, for example, that an AP2 transcription factor is a transcription factor conserved across the phylum Apicomplexa and that a DNA region to which the AP2 transcription factor binds is conserved, that is, a DNA sequence to be bound is thought to be conserved. In fact, as shown in FIG. 4 in PLOS Neglected Tropical Diseases, DOI: 10.1371/journal.pntd. 0004983 Nov.

10, 2016; FIG. 1 in PNAS Jun. 17, 2008, vol. 105 no. 248393; and FIG. 3 in PLOS Neglected Tropical Diseases|DOI: 10.1371/journal.pntd.0003933 Aug. 14, 2015, an AP2-DNA binding domain for a babesial AP2 shows high homology to that for an AP2 transcription factor of Plasmodium spp. Since it has been shown that all of AP2 transcription factors of Plasmodium spp., Toxoplasma spp., and Cryptosporidium spp., which belong to the same phylum Apicomplexa, bind to the sequence that can be inhibited by the AP2-PIPA1 (WGCWWG (SEQ ID NO: 15) (W may be either A or T) and a babesial AP2 domain is conserved, a babesial AP2 is also thought to bind to a sequence that can be inhibited by the AP2-PIPA1.

Based on the above, AP2-PIPA1, which was designed in the same manner as in Example 1, is used to verify an inhibitory effect of a transcription factor in Babesia spp. Inhibitory effect and mouse infection experiments are performed in the same manner as in Example 1. Thus, a concentration of the AP2-PIPA1 that has a therapeutic effect on babesiosis is determined.

### Example 7: Inhibition of Transcription Factor Using PIPA in Theileria Spp

The AP2-PIPA1 is expected to inhibit Theileria spp., which belongs to the same phylum Apicomplexa as Plasmodium spp. The rationale is, for example, that an AP2 transcription factor is a transcription factor conserved across the phylum Apicomplexa and that a DNA region to which the AP2 transcription factor binds is conserved, that is, a DNA sequence to be bound is thought to be conserved. In fact, as shown in FIG. 4 in PLOS Neglected Tropical Diseases, DOI: 10.1371/journal.pntd.0004983 Nov. 10, 2016; FIG. 1 in PNAS Jun. 17, 2008, vol. 105 no. 248393; and FIG. 3 in PLOS Neglected Tropical Diseases|DOI: 10.1371/journal.pntd. 0003933 Aug. 14,

2015, an AP2-DNA binding domain for a theilerial AP2 shows high homology to that for an AP2 transcription factor of Plasmodium spp. Since it has been shown that all of AP2 transcription factors of Plasmodium spp., Toxoplasma spp., and Cryptosporidium spp., which belong to the same phylum Apicomplexa, bind to the sequence that can be inhibited by the AP2-PIPA1 (WGCWWG (SEQ ID NO: 15) (W may be either A or T) and a theilerial AP2 domain is conserved, a theilerial AP2 is also thought to bind to a sequence that can be inhibited by the AP2-PIPA1.

Based on the above, AP2-PIPA1, which was designed in the same manner as in Example 1, is used to verify an inhibitory effect of a transcription factor in Theileria spp. Inhibitory effect and mouse infection experiments are performed in the same manner as in Example 1. Thus, a concentration of the AP2-PIPA1 that has a therapeutic effect on equine piroplasmosis is determined.

### Example 8: Inhibition of Transcription Factor Using PIPA in Other Protozoa in Phylum Apicomplexan

It is believed that all of AP2 transcription factors of protozoa that belong to the phylum Apicomplexa (e.g., Cystoisospora spp.) bind to the sequence that can be inhibited by the AP2-PIPA1 (WGCWWG (SEQ ID NO: 15) (W may be either A or T). Specifically, the AP2-PIPA1 can exert a common inhibitory effect on the phylum Apicomplexa. In fact, one of the most probable sequences recognized by AP2 transcription factors in the phylum Apicomplexa including Plasmodium spp., Toxoplasma spp., Cryptosporidium spp., and a coccidium is reported as TGCATGCA (Pathogens 2019,

8, 47; doi: 10.3390/pathogens8020047), which indicates a high commonality. This sequence includes a sequence that is 100% identical to the sequence (WGCWWG (SEQ ID NO: 15) (W may be either A or T) that can be inhibited by the AP2-PIPA1.

Based on the above, AP2-PIPA1, which was designed in the same manner as in Example 1, is used to verify an inhibitory effect of a transcription factor in other protozoa in phylum Apicomplexan (e.g., Cystoisospora spp.). Inhibitory effect and mouse infection experiments are performed in the same manner as in Example 1. Thus, a concentration of the AP2-PIPA1 that has a therapeutic effect on infection with other protozoa in phylum Apicomplexan (e.g., Cystoisospora spp.) is determined.

### Example 9: Another Example of PIPA Structure for Plasmodium Spp

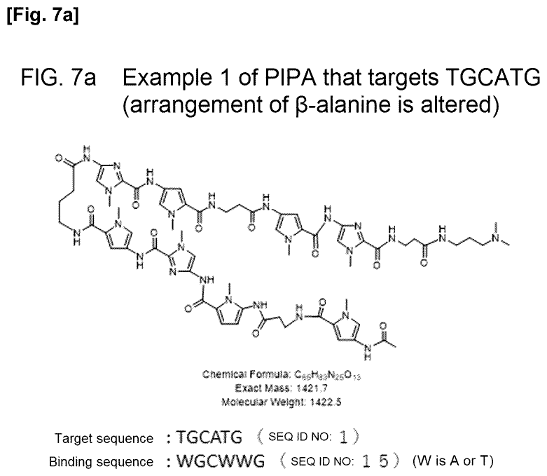

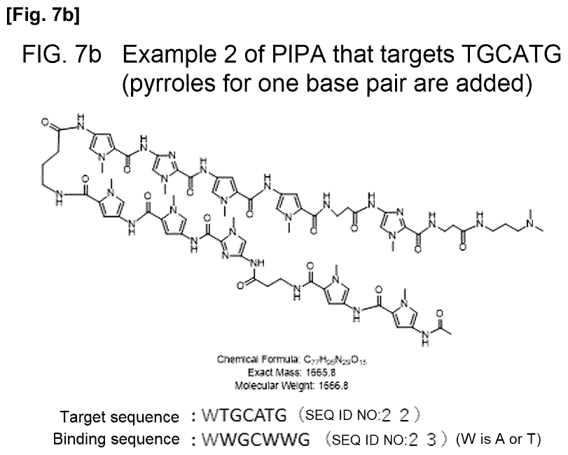

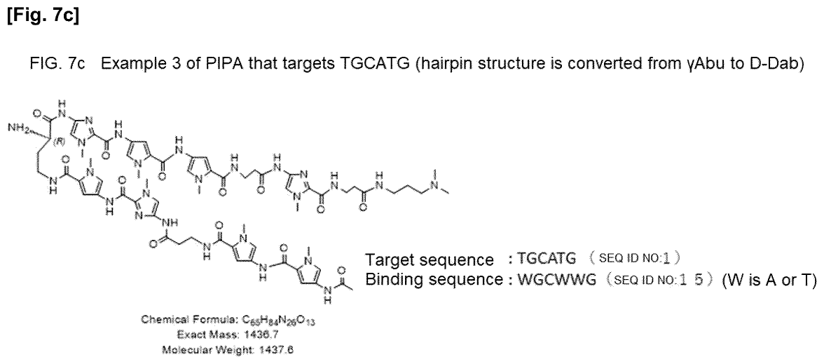

Other PIPAs that bind to the same target sequence (AP2) as in Example 1 were designed. These are shown in FIGS. 7a to 7c. These PIPAs are used to verify an inhibitory effect of a transcription factor in protozoa such as Plasmodium spp. in the same manner as in Example 1. Inhibitory effect and mouse infection experiments are performed in the same manner as in Example 1. Thus, a concentration of the AP2-PIPA1 that has a therapeutic effect on infection with protozoa such as Plasmodium spp. is determined.

### Example 10: Design of PIPA Targeting Another Sequence in Plasmodium Spp

ChIP-seq analysis was performed to search for a binding sequence for a plasmodial transcription factor, yielding TGCACA (SEQ ID NO: 19). A PIPA was designed in the same manner as Example 1, and verified for an inhibitory effect of a transcription factor with a PIPA targeting TGCACA (SEQ ID NO: 19) by a mouse infection experiment, etc.

### Example 11: Preparation Example of Therapeutic Drug Against Malaria

A PIPA is considered a drug discovery modality without requiring a DDS due to its high cell transfection efficiency. Therefore, when AP2-PIPA1 is formulated, it is formulated in the same manner as for an ordinary drug.

Examples of ordinary formulations include a pill, a capsule, a granule, a powder, and a solution for oral preparation; an ointment, a patch, and a lotion for external preparation; and a solution for injection, as well as an eye drop, a nasal drop, a suppository, and an inhalant. AP2-PIPA1 is mixed with such a formulation and administered to a patient by a routine administration method.

### Example 12: In Vivo Test

AP2-PIPA1, which was designed in the same manner as in Example 1, was used to verify an inhibitory effect in vivo. Mice wherein red blood cells had been replaced with human red blood cells were infected with an artemisinin-resistant plasmodium and a therapeutic effect was examined in the thus-infected mice. The effect was verified in the case of administration of a PIPA alone and the case of combined administration with artemisinin.

Humanized mice were created by injecting human red blood cells (RBCs) into NOG-SCID mice. One milliliter of 50% HCT, A+ve red blood cells prepared with 0.5% Albumax and 3.1 mM hypoxanthine was injected into the NOG-SCID mice once daily for 3 weeks until 80% of the human red blood cells were established. The humanized mice were then infected with an artemisinin-resistant plasmodium (Pf lek122) by 4×10⁷ RBCS infected with synchronized ring-stage parasites, and checked for parasitemia by determining a rate of Giemsa-stain positive RBCs from the infected mice. On days 21, 22, 23, 24, and 25, AP2-PIPA1 (AP2-1), artemisinin, or both were injected i.p. at the indicated dose.

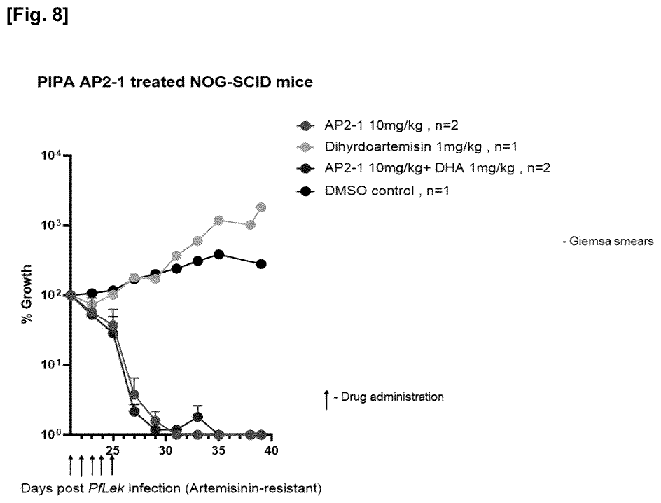

The results are shown in FIG. 8. As can be seen from this figure, an effect of artemisinin could not be observed, but a therapeutic effect on malaria infection was confirmed in both cases of administration of a PIPA alone and of combined administration with artemisinin.

### Example 13: Drug Delivery Study with Conjugate

A conjugate of AP2-PIPA1, which is designed in the same manner as in Example 1, with a compound which specifically binds to a protozoan protein is produced, and a drug delivery effect of this conjugate is verified.

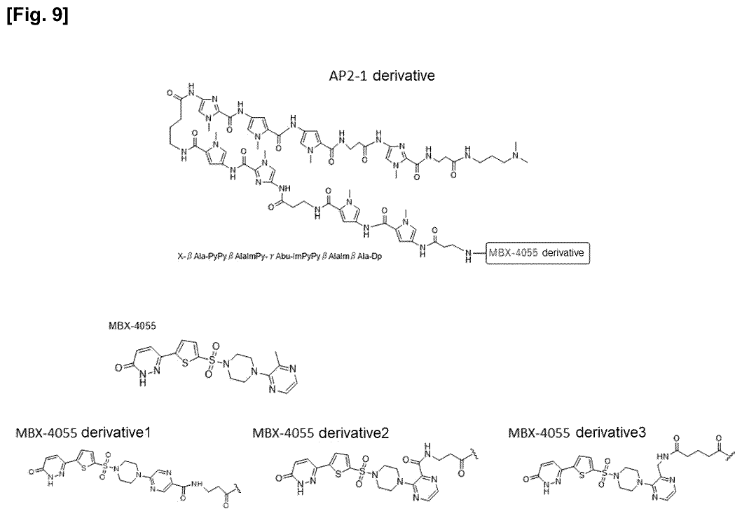

MBX-4055 and its derivatives, which bind to a protein on a surface of a malaria-infected red blood cell (expressed early in infection), are used as the compound which specifically binds to a protozoan protein (FIG. 9). Conjugates of AP2-PIPA1 with MBX-4055 and AP2-PIPA1 with an MBX-4055 derivative are produced to verify for a drug delivery effect, and a transcription factor inhibitory effect and a therapeutic effect with the conjugates.

### (Notes)

Although the present disclosure has been illustrated with reference to preferred embodiments of the present disclosure, it is understood that the scope of the present disclosure should be construed only by the claims. It is to be understood that the patents, patent applications, and other references cited herein should be incorporated herein by reference in its entirety as if the contents themselves are specifically set forth herein. This application claims priority to Japanese Patent Application No. 2022-67631 filed on Apr. 15, 2022 with the Japan Patent Office, the contents of which are incorporated herein by reference in their entirety as if entire contents constitute contents herein.

### INDUSTRIAL APPLICABILITY

The present disclosure can provide an antiprotozoal drug that targets a protozoan transcription factor by using a pyrrole imidazole polyamide (PIPA) and thus enables drug discovery for protozoan infections for which no radical therapeutic drugs have been developed. Therefore, it is expected to be applied in the medical field.

### SEQUENCE LISTING FREE TEXT

SEQ ID NOS: 1 to 7: Binding regions of protozoan transcription factors to which a PIPA according to one embodiment of the present disclosure specifically binds.
SEQ ID NOs: 8 to 14: Binding regions of protozoan transcription factors to which a PIPA according to another embodiment of the present disclosure specifically binds.
SEQ ID NOs: 15 to 23: Target and binding sequences of a PIPA according to one embodiment

1. A pyrrole imidazole polyamide (PIPA) which specifically binds to a binding region for a protozoan transcription factor.

2. The pyrrole imidazole polyamide (PIPA) which specifically binds to a binding region for a protozoan transcription factor according to claim 1, wherein the protozoan transcription factor is a transcription factor specific to a protozoon.

3. The PIPA according to claim 1 or 2, wherein the PIPA functions as a pseudo-transcription factor.

4. The PIPA according to any one of claims 1 to 3, wherein the PIPA has a binding affinity of about 500 nM or less as a dissociation constant (Kd value) for the binding region.

5. The PIPA according to any one of claims 1 to 4, wherein the PIPA has a hairpin structure or a cyclic structure, or two linear PIPAs are used in combination.

6. The PIPA according to any one of claims 1 to 5, wherein the PIPA inhibits a morphological change of the protozoon to at least a gametocyte.

7. The PIPA according to any one of claims 1 to 6, wherein the transcription factor comprises an AP2 family transcription factor.

8. The PIPA according to any one of claims 1 to 7, wherein the binding region comprises 5′-TGCATG-3′ (SEQ ID NO: 1) or an altered sequence thereof, the altered sequence comprising a sequence wherein any one base in 5′-TGCATG-3′ (SEQ ID NO: 1) is deleted or mutated and a sequence wherein one base is added at any position in 5′-TGCATG-3′ (SEQ ID NO: 1).

9. The PIPA according to any one of claims 1 to 8, wherein the binding region comprises NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.

10. The PIPA according to any one of claims 1 to 9, wherein the binding region comprises 5′-TGCACT-3′ (SEQ ID NO: 8) or an altered sequence thereof, the altered sequence comprising a sequence wherein any one base in 5′-TGCACT-3′ (SEQ ID NO: 8) is deleted or mutated and a sequence wherein one base is added at any position in 5′-TGCACT-3′ (SEQ ID NO: 8).

11. The PIPA according to any one of claim 1 to 7 or 10, wherein the binding region comprises NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.

12. The PIPA according to any one of claims 1 to 11, wherein the PIPA comprises the following structure: 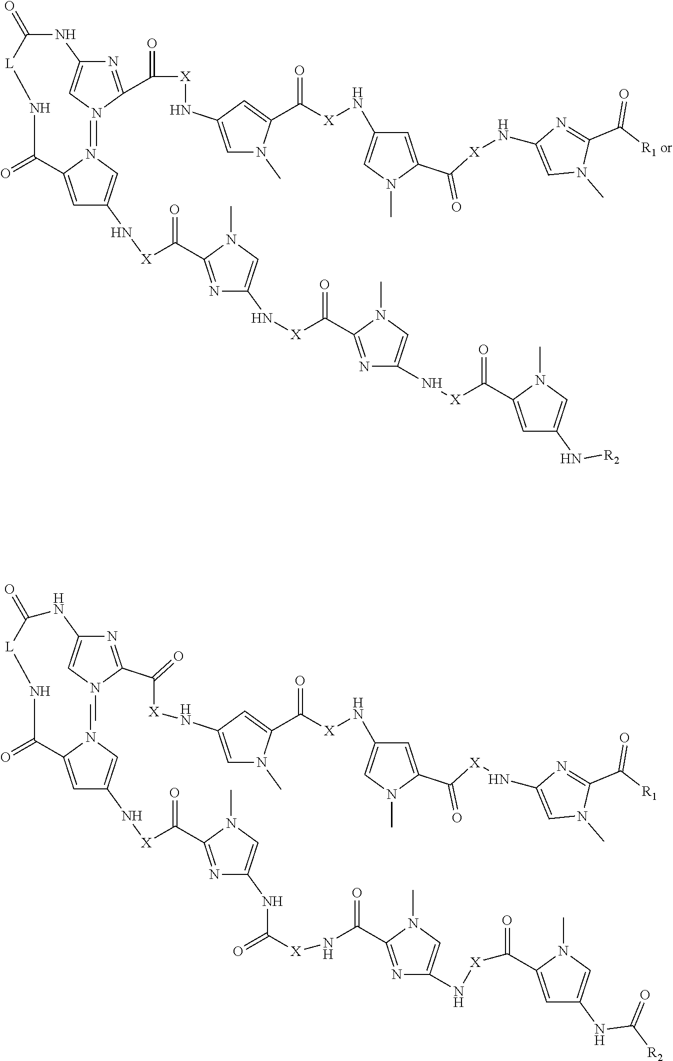
wherein
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.

13. The PIPA according to claim 12, wherein the aliphatic amino acid residue comprises a molecule having an amino group and a carboxy group.

14. The PIPA according to claim 12 or 13, wherein the aliphatic amino acid residue comprises glycine, β-alanine, γ-aminobutyric acid, R2,4-diaminobutyric acid, and 5-aminovaleric acid.

15. The PIPA according to any one of claims 12 to 14, wherein the PIPA has the following structure:

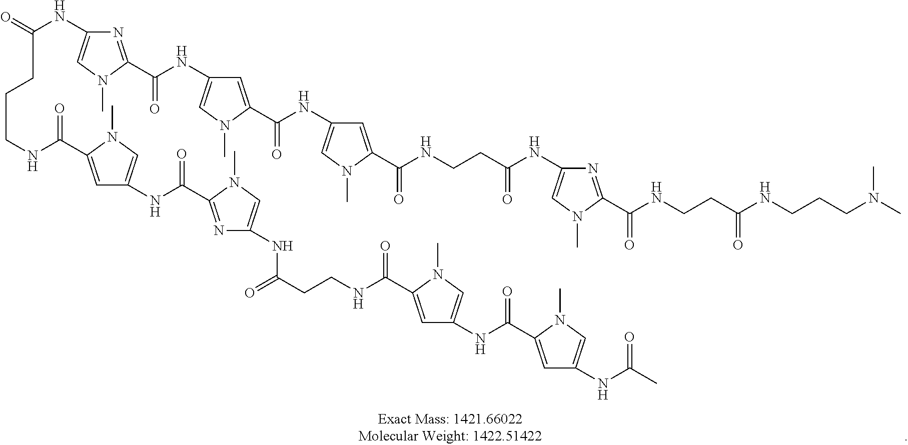 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

16. The PIPA according to any one of claims 12 to 15, wherein the PIPA has the following structure:

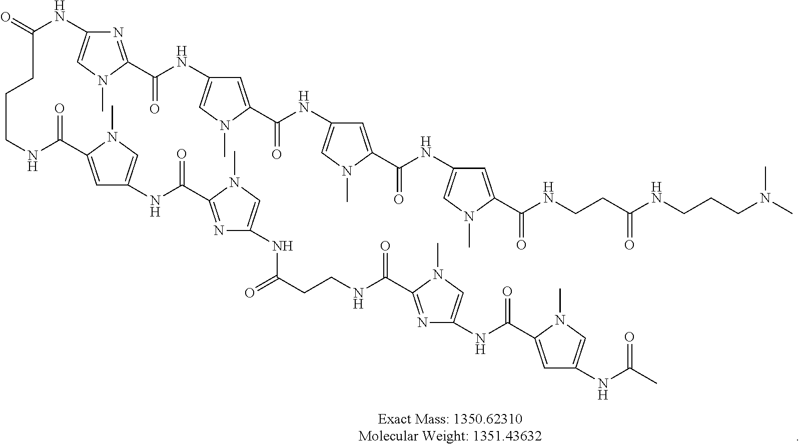 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

17. The PIPA according to any one of claims 1 to 16, wherein the protozoon comprises Plasmodium spp., Leishmania spp., Toxoplasma spp., Cryptosporidium spp., and a coccidium.

18. A pyrrole imidazole polyamide (PIPA) which specifically binds to a binding region for a protozoan transcription factor, for inhibiting a function of the protozoan transcription factor.

19. The PIPA according to claim 18, wherein the protozoan transcription factor is a transcription factor specific to a protozoon.

20. The PIPA according to claim 18 or 19, wherein the PIPA functions as a pseudo-transcription factor.

21. The PIPA according to any one of claims 18 to 20, wherein the PIPA has a binding affinity of about 500 nM or less as a dissociation constant (Kd value) for the binding region.

22. The PIPA according to any one of claims 18 to 21, wherein the PIPA has a hairpin structure or a cyclic structure, or two linear PIPAs are used in combination.

23. The PIPA according to any one of claims 18 to 22, wherein the PIPA inhibits a morphological change of the protozoon to at least a gametocyte.

24. The PIPA according to any one of claims 18 to 23, wherein the transcription factor comprises an AP2 family transcription factor.

25. The PIPA according to any one of claims 18 to 24, wherein the binding region comprises 5′-TGCATG-3′ (SEQ ID NO: 1) or an altered sequence thereof, the altered sequence comprising a sequence wherein any one base in 5′-TGCATG-3′ (SEQ ID NO: 1) is deleted or mutated and a sequence wherein one base is added at any position in 5′-TGCATG-3′ (SEQ ID NO: 1).

26. The PIPA according to any one of claims 18 to 25, wherein the binding region comprises NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.

27. The PIPA according to any one of claims 18 to 26, wherein the binding region comprises 5′-TGCACT-3′ (SEQ ID NO: 8) or an altered sequence thereof, the altered sequence comprising a sequence wherein any one base in 5′-TGCACT-3′ (SEQ ID NO: 8) is deleted or mutated and a sequence wherein one base is added at any position in 5′-TGCACT-3′ (SEQ ID NO: 8).

28. The PIPA according to any one of claim 18 to 24 or 27, wherein the binding region comprises NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.

29. The PIPA according to any one of claims 18 to 28, wherein the PIPA comprises the following structure: 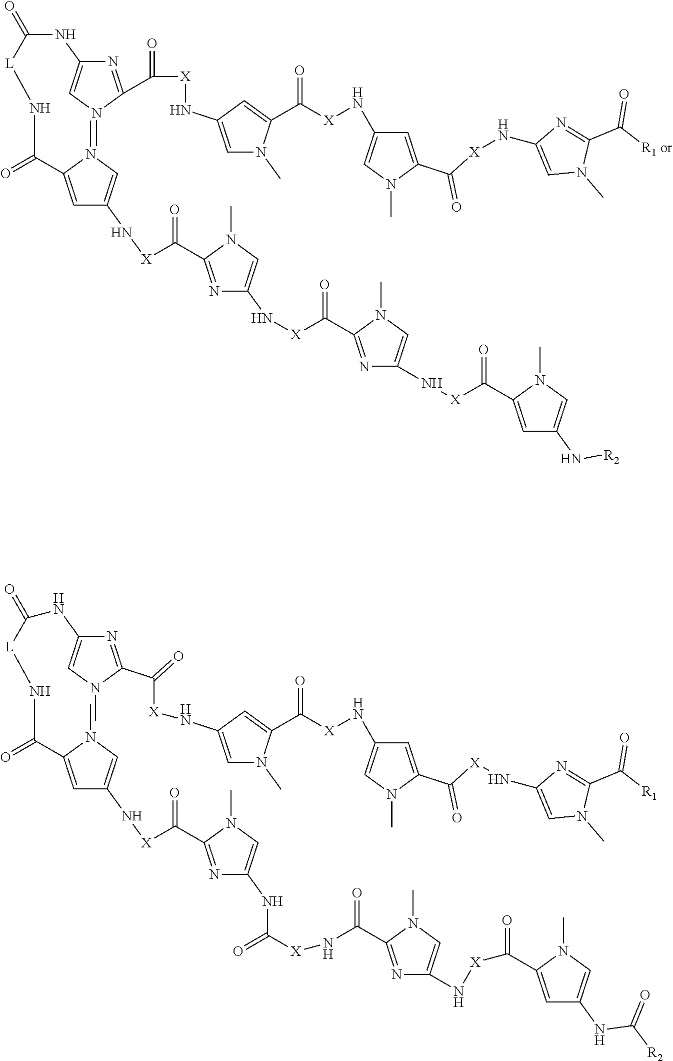
where
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.

30. The PIPA according to claim 29, wherein the aliphatic amino acid residue comprises a molecule having an amino group and a carboxy group.

31. The PIPA according to claim 29 or 30, wherein the aliphatic amino acid residue comprises glycine, β-alanine, γ-aminobutyric acid, R2,4-diaminobutyric acid, and 5-aminovaleric acid.

32. The PIPA according to any one of claims 29 to 31, wherein the PIPA has the following structure:

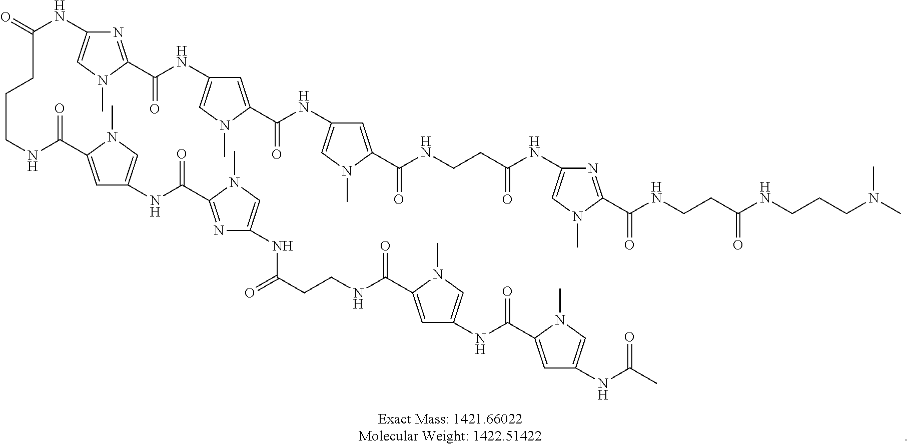 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

33. The PIPA according to any one of claims 29 to 31, wherein the PIPA has the following structure:

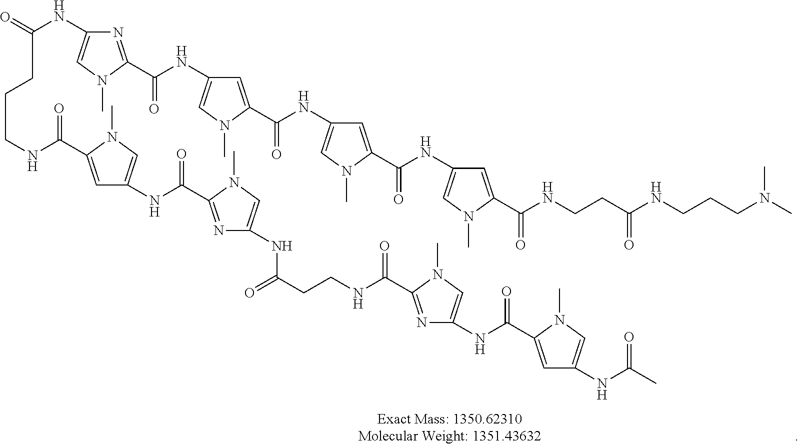 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

34. The PIPA according to any one of claims 18 to 33, wherein the protozoon comprises Plasmodium spp., Leishmania spp., Toxoplasma spp., Cryptosporidium spp., and a coccidium.

35. A pyrrole imidazole polyamide (PIPA) which specifically binds to a binding region for a protozoan transcription factor, for use as a pseudo-transcription factor.

36. A composition comprising
a pyrrole imidazole polyamide (PIPA) which specifically binds to a binding region for a protozoan transcription factor, for inhibiting a function of the protozoan transcription factor.

37. The composition according to claim 36, wherein the protozoan transcription factor is a transcription factor specific to a protozoon.

38. The composition according to claim 36 or 37, wherein the composition functions as a pseudo-transcription factor.

39. The composition according to any one of claims 36 to 38, wherein the composition has a binding affinity of about 500 nM or less as a dissociation constant (Kd value) for the binding region.

40. The composition according to any one of claims 36 to 39, wherein the PIPA has a hairpin structure or a cyclic structure, or two linear PIPAs are used in combination.

41. The composition according to any one of claims 36 to 40, wherein the composition inhibits a morphological change of the protozoon to at least a gametocyte.

42. The composition according to any one of claims 36 to 41, wherein the transcription factor comprises an AP2 family transcription factor.

43. The composition according to any one of claims 36 to 42, wherein the binding region comprises 5′-TGCATG-3′ (SEQ ID NO: 1, or an altered sequence thereof, the altered sequence comprising a sequence wherein any one base in 5′-TGCATG-3′ (SEQ ID NO: 1) is deleted or mutated and a sequence wherein one base is added at any position in 5′-TGCATG-3′ (SEQ ID NO: 1).

44. The composition according to any one of claims 36 to 43, wherein the binding region comprises NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.

45. The composition according to any one of claims 36 to 44, wherein the binding region comprises 5′-TGCACT-3′ (SEQ ID NO: 8, or an altered sequence thereof, the altered sequence comprising a sequence wherein any one base in 5′-TGCACT-3′ (SEQ ID NO: 8) is deleted or mutated and a sequence wherein one base is added at any position in 5′-TGCACT-3′ (SEQ ID NO: 8).

46. The composition according to any one of claim 36 to 42 or 45, wherein the binding region comprises NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.

47. The composition according to any one of claims 36 to 46, wherein the PIPA comprises the following structure: 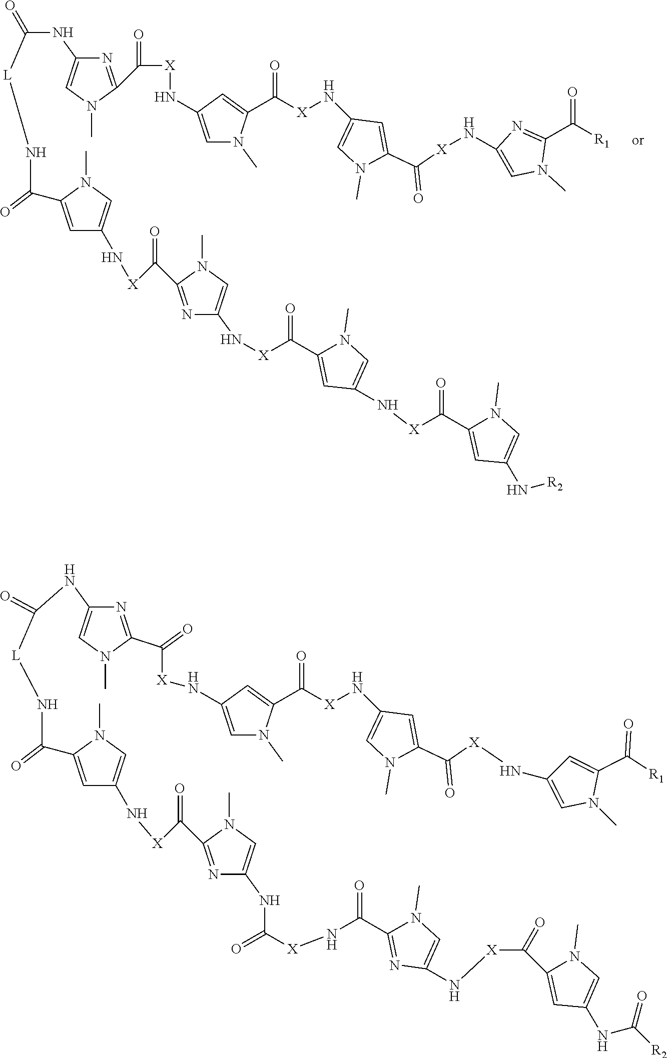
wherein
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.

48. The composition according to claim 47, wherein the aliphatic amino acid residue comprises a molecule having an amino group and a carboxy group.

49. The composition according to claim 47 or 48, wherein the aliphatic amino acid residue comprises glycine, β-alanine, γ-aminobutyric acid, R2,4-diaminobutyric acid, and 5-aminovaleric acid.

50. The composition according to any one of claims 47 to 49, wherein the PIPA has the following structure:

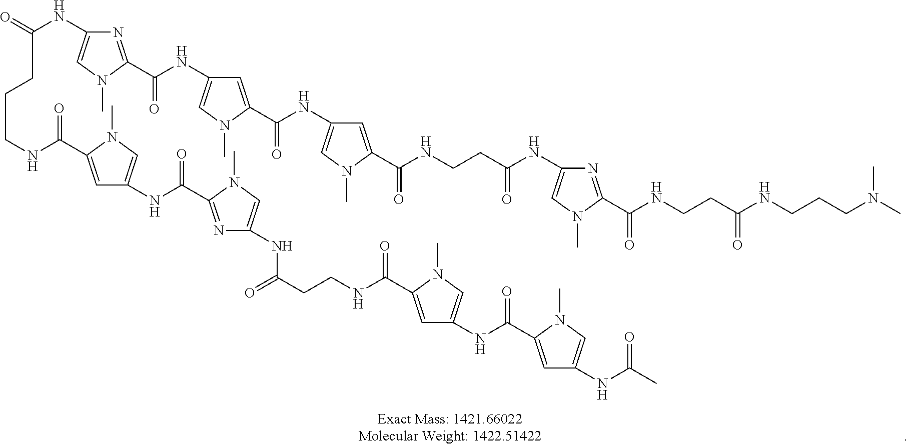 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

51. The composition according to any one of claims 47 to 49, wherein the PIPA has the following structure:

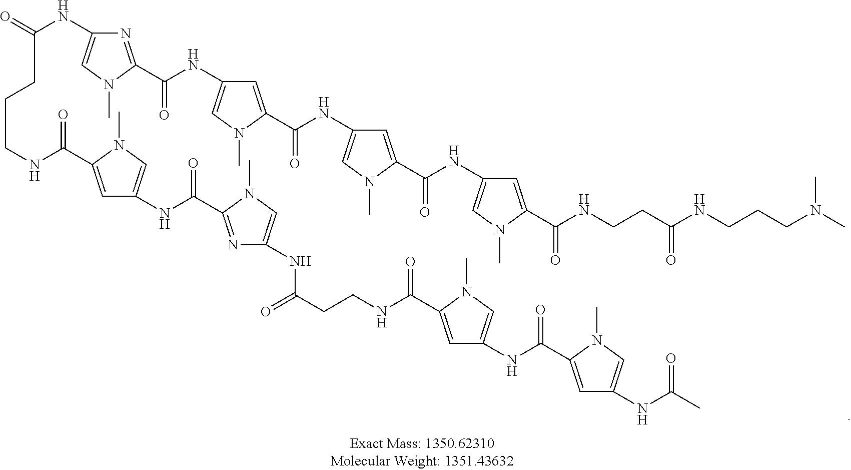 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n(C)c2)n(C)c1]

52. The composition according to any one of claims 36 to 51, wherein the protozoon comprises Plasmodium spp., Leishmania spp., Toxoplasma spp., Cryptosporidium spp., and a coccidium.

53. A composition comprising
a pyrrole imidazole polyamide (PIPA) which specifically binds to a binding region for a protozoan transcription factor, for use as a pseudo-transcription factor.

54. A protozoan transcription factor inhibitor comprising
a pyrrole imidazole polyamide (PIPA) which specifically binds to a binding region for a protozoan transcription factor.

55. The protozoan transcription factor inhibitor according to claim 54, wherein the protozoan transcription factor is a transcription factor specific to a protozoon.

56. The protozoan transcription factor inhibitor according to claim 54 or 55, wherein the protozoan transcription factor inhibitor functions as a pseudo-transcription factor.

57. The protozoan transcription factor inhibitor according to any one of claims 54 to 56, wherein the protozoan transcription factor inhibitor has a binding affinity of about 500 nM or less as a dissociation constant (Kd value) for the binding region.

58. The protozoan transcription factor inhibitor according to any one of claims 54 to 57, wherein the PIPA has a hairpin structure or a cyclic structure, or two linear PIPAs are used in combination.

59. The protozoan transcription factor inhibitor according to any one of claims 54 to 58, wherein the protozoan transcription factor inhibitor inhibits a morphological change of the protozoon to at least a gametocyte.

60. The protozoan transcription factor inhibitor according to any one of claims 54 to 59, wherein the transcription factor comprises an AP2 family transcription factor.

61. The protozoan transcription factor inhibitor according to any one of claims 54 to 60, wherein the binding region comprises 5′-TGCATG-3′ (SEQ ID NO: 1) or an altered sequence thereof, the altered sequence comprising a sequence wherein any one base in 5′-TGCATG-3′ (SEQ ID NO: 1) is deleted or mutated and a sequence wherein one base is added at any position in 5′-TGCATG-3′ (SEQ ID NO: 1).

62. The protozoan transcription factor inhibitor according to any one of claims 54 to 61, wherein the binding region comprises NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.

63. The protozoan transcription factor inhibitor according to any one of claims 54 to 62, wherein the binding region comprises 5′-TGCACT-3′ (SEQ ID NO: 8) or an altered sequence thereof, the altered sequence comprising a sequence wherein any one base in 5′-TGCACT-3′ (SEQ ID NO: 8) is deleted or mutated and a sequence wherein one base is added at any position in 5′-TGCACT-3′ (SEQ ID NO: 8).

64. The protozoan transcription factor inhibitor according to any one of claim 54 to 60 or 63, wherein the binding region comprises NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.

65. The protozoan transcription factor inhibitor according to any one of claims 54 to 64, wherein the PIPA comprises the following structure: 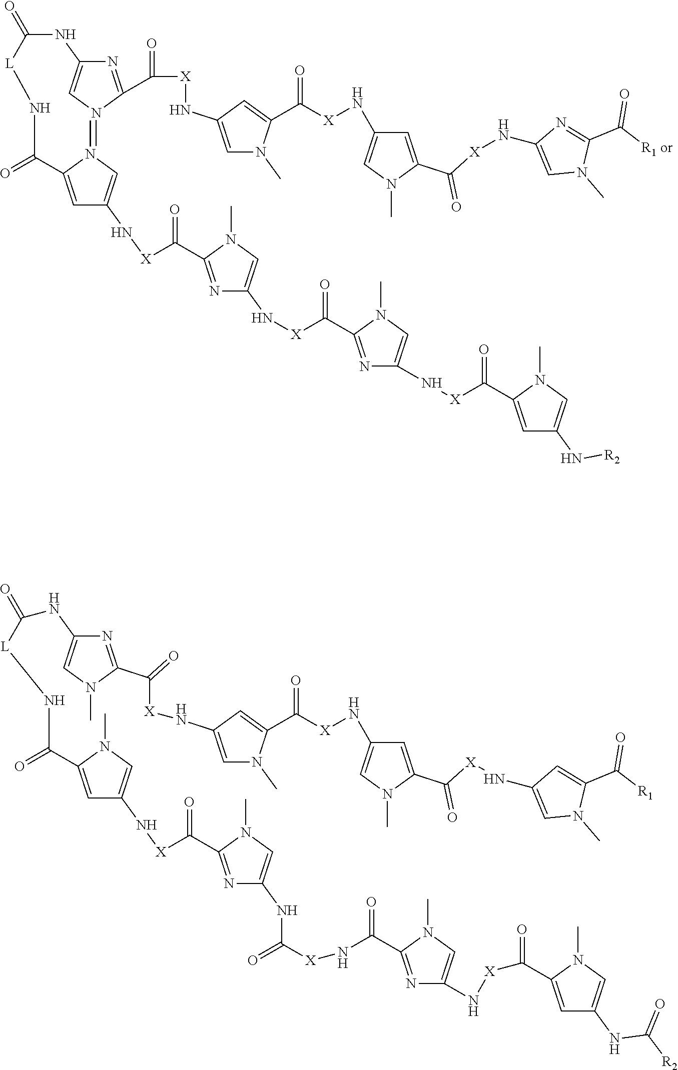
wherein
L is a C2-6 alkyl linker;
R₁ and R₂ are optimally substituted alkyl, R₁ and R₂ may be taken together with each other to form a C2-6 alkyl linker; and
X is a bond or an aliphatic amino acid residue.

66. The protozoan transcription factor inhibitor according to claim 65, wherein the aliphatic amino acid residue comprises a molecule having an amino group and a carboxy group.

67. The protozoan transcription factor inhibitor according to claim 65 or 66, wherein the aliphatic amino acid residue comprises glycine, β-alanine, γ-aminobutyric acid, R2,4-diaminobutyric acid, and 5-aminovaleric acid.

68. The protozoan transcription factor inhibitor according to any one of claims 65 to 67, wherein the PIPA has the following structure:

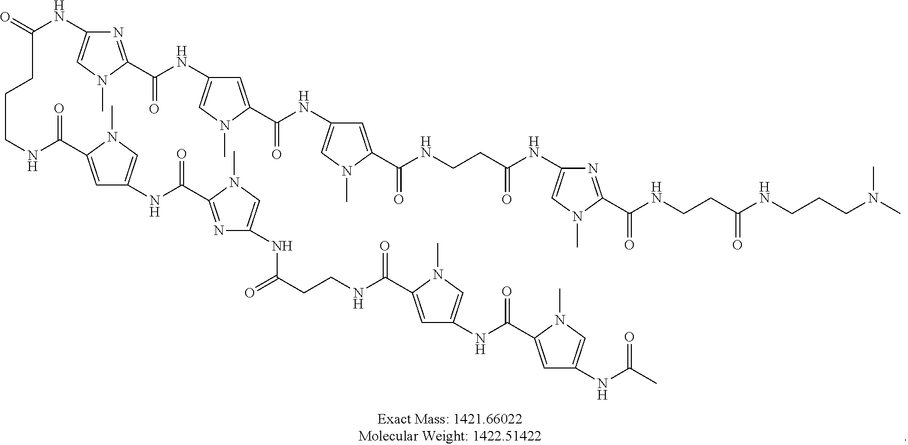 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

69. The protozoan transcription factor inhibitor according to any one of claims 65 to 68, wherein the PIPA has the following structure:

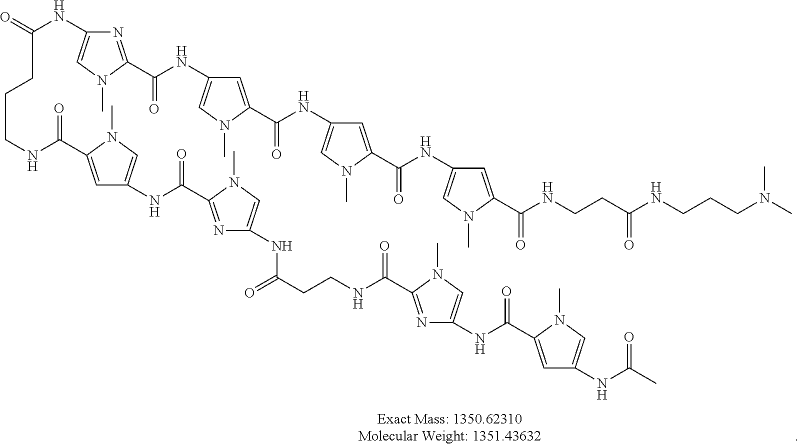 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

70. The protozoan transcription factor inhibitor according to any one of claims 54 to 69, wherein the protozoon comprises Plasmodium spp., Leishmania spp., Toxoplasma spp., Cryptosporidium spp., and a coccidium.

71. A method for producing a protozoan transcription factor inhibitor comprising a pyrrole imidazole polyamide (PIPA), the method comprising:
providing a binding region for a protozoan transcription factor; and
designing the PIPA to specifically bind to the binding region.

72. The method according to claim 71, wherein the designing comprises linking a pyrrole and/or an imidazole selected to correspond to a nucleotide sequence of the binding region and, if necessary, replacing one or more pyrroles or imidazoles in the linked pyrrole and/or imidazole molecule with β-alanine.

73. A therapeutic or prophylactic agent for a disease caused by a protozoon, the agent comprising
a protozoan transcription factor comprising a pyrrole imidazole polyamide (PIPA) which specifically binds to a binding region for the protozoan transcription factor.

74. The therapeutic or prophylactic agent according to claim 73, wherein the protozoan transcription factor is a transcription factor specific to a protozoon.

75. The therapeutic or prophylactic agent according to claim 73 or 74, wherein the therapeutic or prophylactic agent functions as a pseudo-transcription factor.

76. The therapeutic or prophylactic agent according to any one of claims 73 to 75, wherein the therapeutic or prophylactic agent has a binding affinity of about 500 nM or less as a dissociation constant (Kd value) for the binding region.

77. The therapeutic or prophylactic agent according to any one of claims 73 to 76, wherein the PIPA has a hairpin structure or a cyclic structure, or two linear PIPAs are used in combination.

78. The therapeutic or prophylactic agent according to any one of claims 73 to 77, wherein the therapeutic or prophylactic agent inhibits a morphological change of the protozoon to at least a gametocyte.

79. The therapeutic or prophylactic agent according to any one of claims 73 to 78, wherein the transcription factor comprises an AP2 family transcription factor.

80. The therapeutic or prophylactic agent according to any one of claims 73 to 79, wherein the binding region comprises 5′-TGCATG-3′ (SEQ ID NO: 1) or an altered sequence thereof, the altered sequence comprising a sequence wherein any one base in 5′-TGCATG-3′ (SEQ ID NO: 1) is deleted or mutated and a sequence wherein one base is added at any position in 5′-TGCATG-3′ (SEQ ID NO: 1).

81. The therapeutic or prophylactic agent according to any one of claims 73 to 80, wherein the binding region comprises NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.

82. The therapeutic or prophylactic agent according to any one of claims 73 to 81, wherein the binding region comprises 5′-TGCACT-3′ (SEQ ID NO: 8) or an altered sequence thereof, the altered sequence comprising a sequence wherein any one base in 5′-TGCACT-3′ (SEQ ID NO: 8) is deleted or mutated and a sequence wherein one base is added at any position in 5′-TGCACT-3′ (SEQ ID NO: 8).

83. The therapeutic or prophylactic agent according to any one of claim 73 to 79 or 82, wherein the binding region comprises NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.

84. The therapeutic or prophylactic agent according to any one of claims 73 to 83, wherein the PIPA comprises the following structure: 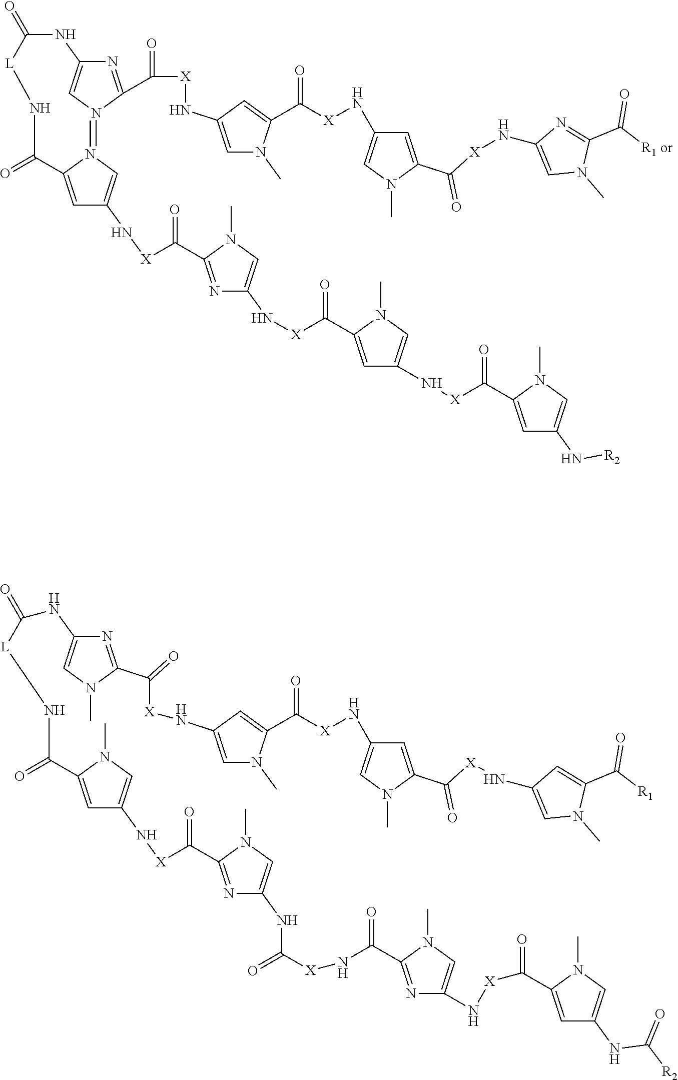
wherein
L is a C2-6 alkyl linker;
R₁ and R₂ are optimally substituted alkyl, R₁ and R₂ may be taken together with each other to form a C2-6 alkyl linker; and
X is a bond or an aliphatic amino acid residue.

85. The therapeutic or prophylactic agent according to claim 84, wherein the aliphatic amino acid residue comprises a molecule having an amino group and a carboxy group.

86. The therapeutic or prophylactic agent according to claim 84 or 85, wherein the aliphatic amino acid residue comprises glycine, β-alanine, γ-aminobutyric acid, R2,4-diaminobutyric acid, and 5-aminovaleric acid.

87. The therapeutic or prophylactic agent according to any one of claims 84 to 86, wherein the PIPA has the following structure:

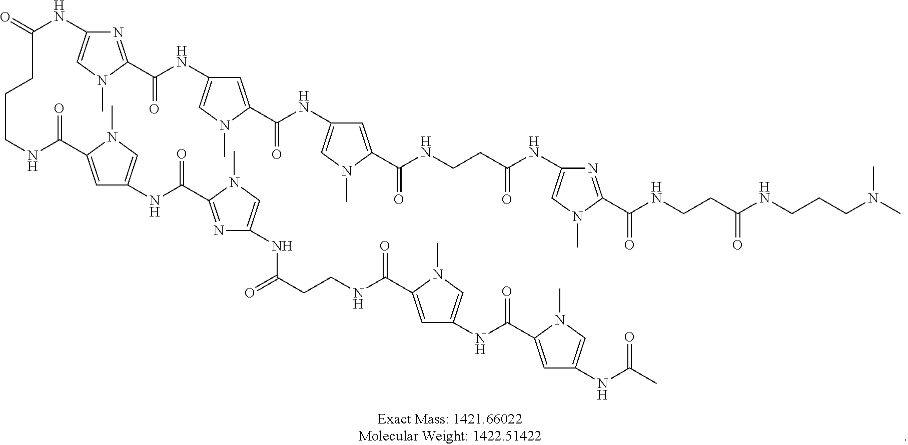 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

88. The therapeutic or prophylactic agent according to any one of claims 84 to 86, wherein the PIPA has the following structure:

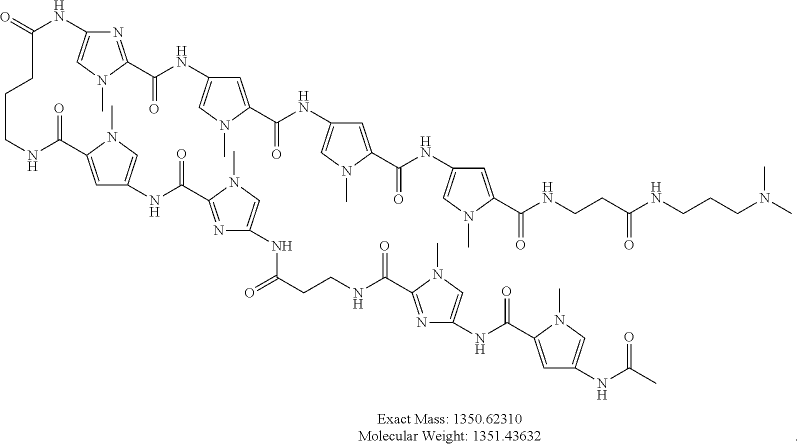 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

89. The therapeutic or prophylactic agent according to any one of claims 73 to 88, wherein the protozoon comprises Plasmodium spp., Leishmania spp., Toxoplasma spp., Cryptosporidium spp., and a coccidium.

90. A pyrrole imidazole polyamide (PIPA) which specifically binds to a binding region for a protozoan transcription factor, for treating or preventing a disease caused by a protozoon.

91. A conjugate comprising:
a pyrrole imidazole polyamide (PIPA) which specifically binds to a binding region for a protozoan transcription factor; and
a protozoon-specific factor that is different from the PIPA.

92. The conjugate according to claim 91, wherein the protozoon-specific factor comprises a factor that binds to a surface protein of a malaria-infected red blood cell.

93. The conjugate according to claim 91, wherein the protozoon-specific factor has a proliferation inhibitory effect on Plasmodium spp., Leishmania spp., Toxoplasma spp., Cryptosporidium spp., and/or a coccidium.

94. The conjugate according to any one of claims 91 to 93, wherein the PIPA and the protozoon-specific factor are linked by a linker.

95. The conjugate according to claim 94, wherein the linker is a C1-6 alkyl linker.

96. The conjugate according to any one of claims 91 to 93, wherein the PIPA and the protozoon-specific factor are directly linked.

97. The conjugate according to claim 91, wherein the protozoon-specific factor comprises a pyridazinone derivative.

98. The conjugate according to claim 97, wherein the pyridazinone derivative is MBX-4055 represented by the following formula:

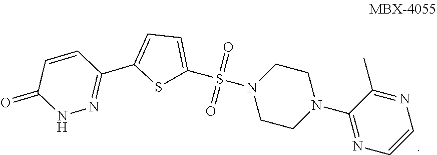 [SMILES: Cc1nccnc1N1CCN(S(=O)(=O)c2ccc(-c3ccc(=O)[nH]n3)s2)CC1]

99. The conjugate according to claim 97, wherein the pyridazinone derivative is an MBX-4055 derivative represented by the following formula:

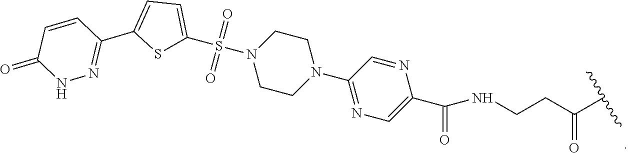 [SMILES: *C(=O)CCNC(=O)c1cnc(N2CCN(S(=O)(=O)c3ccc(-c4ccc(=O)[nH]n4)s3)CC2)cn1]

100. The conjugate according to claim 97, wherein the pyridazinone derivative is an MBX-4055 derivative represented by the following formula:

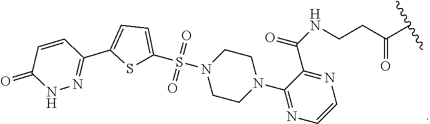 [SMILES: *C(=O)CCNC(=O)c1nccnc1N1CCN(S(=O)(=O)c2ccc(-c3ccc(=O)[nH]n3)s2)CC1]

101. The conjugate according to claim 97, wherein the pyridazinone derivative is an MBX-4055 derivative represented by the following formula:

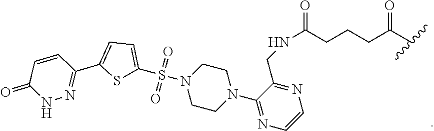 [SMILES: *C(=O)CCCC(=O)NCc1nccnc1N1CCN(S(=O)(=O)c2ccc(-c3ccc(=O)[nH]n3)s2)CC1]

102. The conjugate according to any one of claims 91 to 101, wherein the protozoan transcription factor is a transcription factor specific to a protozoon.

103. The conjugate according to any one of claims 91 to 102, wherein the PIPA functions as a pseudo-transcription factor.

104. The conjugate according to any one of claims 91 to 103, wherein the conjugate has a binding affinity of about 500 nM or less as a dissociation constant (Kd value) for the binding region.

105. The protozoan transcription factor inhibitor according to any one of claims 91 to 104, wherein the PIPA has a hairpin structure or a cyclic structure, or two linear PIPAs are used in combination.

106. The conjugate according to any one of claims 91 to 105, wherein the conjugate inhibits a morphological change of the protozoon to at least a gametocyte.

107. The conjugate according to any one of claims 91 to 106, wherein the transcription factor comprises an AP2 family transcription factor.

108. The conjugate according to any one of claims 91 to 107, wherein the binding region comprises 5′-TGCATG-3′ (SEQ ID NO: 1, or an altered sequence thereof, the altered sequence comprising a sequence wherein any one base in 5′-TGCATG-3′ (SEQ ID NO: 1) is deleted or mutated and a sequence wherein one base is added at any position in 5′-TGCATG-3′ (SEQ ID NO: 1).

109. The conjugate according to any one of claims 91 to 108, wherein the binding region comprises NGCATG (SEQ ID NO: 2), TNCATG (SEQ ID NO: 3), TGNATG (SEQ ID NO: 4), TGCNTG (SEQ ID NO: 5), TGCANG (SEQ ID NO: 6), and TGCATN (SEQ ID NO: 7) where N is A, T, G, or C.

110. The conjugate according to any one of claims 91 to 109, wherein the binding region comprises 5′-TGCACT-3′ (SEQ ID NO: 8) or an altered sequence thereof, the altered sequence comprising a sequence wherein any one base in 5′-TGCACT-3′ (SEQ ID NO: 8) is deleted or mutated and a sequence wherein one base is added at any position in 5′-TGCACT-3′ (SEQ ID NO: 8).

111. The conjugate according to any one of claim 91 to 107 or 110, wherein the binding region comprises NGCACT (SEQ ID NO: 9), TNCACT (SEQ ID NO: 10), TGNACT (SEQ ID NO: 11), TGCNCT (SEQ ID NO: 12), TGCANT (SEQ ID NO: 13), and TGCACN (SEQ ID NO: 14) where N is A, T, G, or C.

112. The conjugate according to any one of claims 91 to 111, wherein the PIPA comprises the following structure: 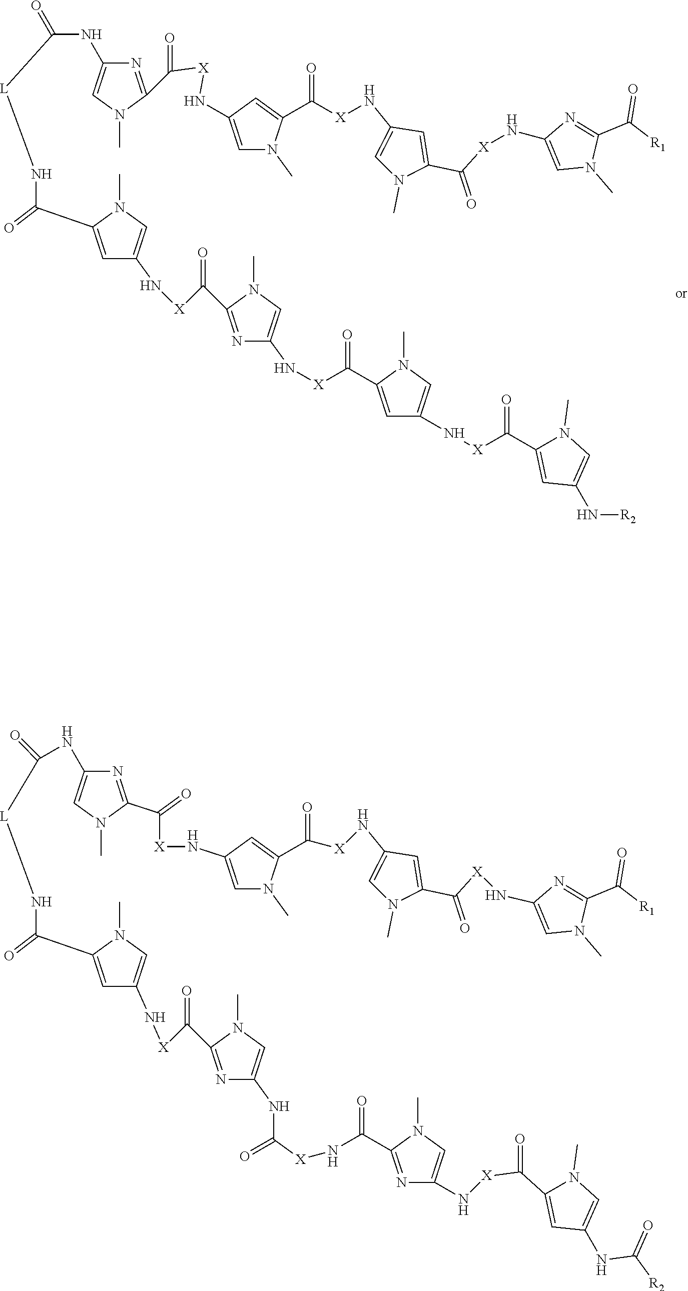
wherein
L is a C2-6 alkyl linker;
R₁ and R₂ are optionally substituted alkyl, R₁ and R₂ may be taken together with each other to form a C2-6 alkyl linker; and
X is a bond or an aliphatic amino acid residue.

113. The conjugate according to claim 112, wherein the aliphatic amino acid residue comprises a molecule having an amino group and a carboxy group.

114. The conjugate according to claim 112 or 113, wherein the aliphatic amino acid residue comprises glycine, β-alanine, γ-aminobutyric acid, R2,4-diaminobutyric acid, and 5-aminovaleric acid.

115. The conjugate according to any one of claims 112 to 114, wherein the PIPA has the following structure:

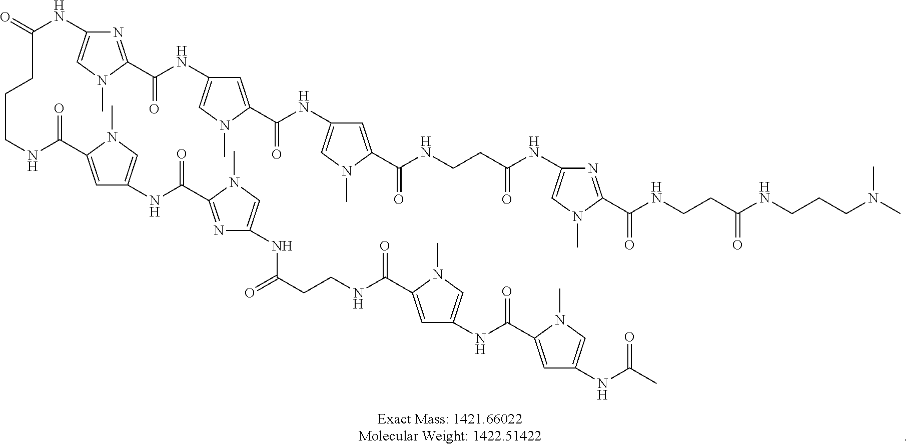 [SMILES: CC(=O)Nc1cc(C(=O)Nc2cc(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)NCCC(=O)Nc8cn(C)c(C(=O)NCCC(=O)NCCCN(C)C)n8)n(C)c7)n(C)c6)n5)n(C)c4)n3)n(C)c2)n(C)c1]

116. The conjugate according to any one of claims 112 to 115, wherein the PIPA has the following structure:

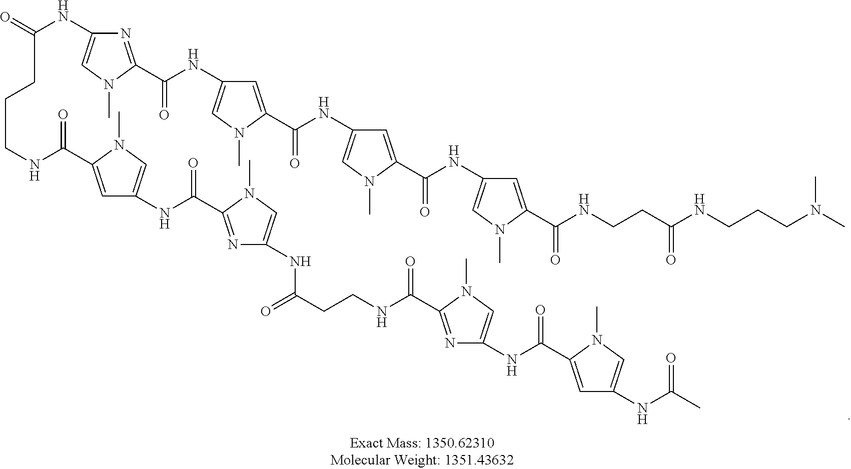 [SMILES: CC(=O)Nc1cc(C(=O)Nc2cn(C)c(C(=O)NCCC(=O)Nc3cn(C)c(C(=O)Nc4cc(C(=O)NCCCC(=O)Nc5cn(C)c(C(=O)Nc6cc(C(=O)Nc7cc(C(=O)Nc8cc(C(=O)NCCC(=O)NCCCN(C)C)n(C)c8)n(C)c7)n(C)c6)n5)n(C)c4)n3)n2)n(C)c1]

117. The conjugate according to any one of claims 91 to 116, wherein the protozoon comprises Plasmodium spp., Leishmania spp., Toxoplasma spp., Cryptosporidium spp., and a coccidium.


SEQUENCE LISTING (23 sequences)

SEQ ID NO: 1-20 - skipped (20 entries)

SEQ ID NO: 21 - DNA, 32 bp, synthetic construct
  misc_feature 1..32: note = Sequence used for the binding analysis in Figure 1b.
  source 1..32: mol_type = other DNA
agtctgcatgagtcttttgactcatgcagact

SEQ ID NO: 22-23 - skipped (2 entries)